(19) United States
(12) Patent Application Publication
(10) Pub. No.: US 2015/0343173 A1
(43) Pub. Date: Dec. 3, 2015

(54) CATHETER OR CANNULA ARRANGEMENT WITH UNIT FOR MONITORING LENGTH OF STAY OF THE SAME IN A BODY

(71) Applicants: BERLINGER & CO. AG, Ganterschwil (CH); STAR SYRINGE LIMITED, London (GB)

(72) Inventors: Corneliu Tobescu, Wilen (CH); Paul Mallins, Verbier (CH); David Michael Bower, West Sussex (GB)

(21) Appl. No.: 14/654,668

(22) PCT Filed: Dec. 23, 2013
(86) PCT No.: PCT/CH2013/000232

§ 371 Date: Jun. 22, 2015

Related U.S. Application Data
(60) Provisional application No. 61/745,687, filed on Dec. 24, 2012.

(30) Foreign Application Priority Data
Dec. 24, 2012 (CH) 2936/12

(51) Int. Cl. A61M 25/00; A61M 25/06

(52) U.S. Cl. CPC A61M 25/0017; A61M 25/0606; A61M 2205/18; A61M 2205/27; A61M 2205/60; A61B 2560/028; A61B 5/01

(57) ABSTRACT

A catheter or cannula arrangement includes a catheter or cannula and a security unit for monitoring a length of stay of the catheter or cannula in a body. The security unit includes a signaling unit, an activation unit, and a timer unit. The signaling unit emits a first signal. The activation detection unit detects a first event and produces a second signal indicative of the first event detection. The timer unit receives the second signal and starts a time duration measurement in reaction thereto, and produces and transmits to the signaling unit a third signal as soon as the measured time duration is reaches a preset time value. The signaling unit emits the first signal in reaction to receiving the third signal. The first event can be an at least partial extraction of a needle from the catheter or cannula.

### BACKGROUND OF THE INVENTION

1. Field of the Invention

The invention relates to the field of medical devices and more specifically to catheter or cannula arrangements and constituents thereof.

2. Description of Related Art

Catheter or cannula arrangements are very frequently used worldwide, for humans as well as for animals, in surgery and in other medical applications. Besides the catheter or cannula, they often include a needle inserted in and removable from the catheter or cannula. Usually, the needle is used to pierce the body and the catheter or cannula is introduced through the piercing.

Other catheter or cannula arrangements such as urinary catheters do not include a needle. Specific urinary catheters, more particularly those known as Foley catheters, have at least two separate channels, a first channel for guiding fluids, mainly urine, out of the body, and a second channel for guiding another fluid into a balloon positioned inside the body, the enlarged balloon holding the inner end of the Foley catheter in place inside the body. At the outer end of the Foley catheter, a valve is usually present for controlling flow into and out of the second channel.

Furthermore, catheters are known which include a temperature sensing element for sensing the temperature inside the body. Such a catheter has an electrical connector, typically a cable with an electrical connector plug, by means of which the sensing element can be electrically connected to an external reading device which processes the signals output by the sensing element and displays the temperature as sensed by the sensing element. Such catheters may find application, e.g., as urinary catheters for an early detection of an inflammation.

### SUMMARY OF THE INVENTION

The invention in most of its aspects originated from the observation that, in rare cases, it may happen that a catheter or cannula remains in a human body for a too long time, in particular for so long that an infection takes place at the catheter or cannula. Significant correlation between infection and indwell time has been demonstrated. In order to prevent such undesirable instances, it has been contemplated to provide means for reliably providing an indication reminding of the need of removing and replacing, respectively, the catheter or cannula. In particular, it has been contemplated to provide means for monitoring a length of stay (“dwell time”) of the catheter or cannula in a body.

There are various aspects to such means and corresponding catheter or cannular arrangements including such means. Means for accomplishing the sought task or tasks may be referred to as a security unit. All the aspects can be viewed and understood as independent aspects which, however, may be combined with each other in any way, e.g., pair-wise or combining three of four (or even more) of them.

In addition, there is a special aspect, which is, like the other aspects, related to catheter or cannula arrangements, but which may be realized in combination with a security unit of a described kind, and thus with one or more of the other aspects, but which may, in general, also be realized without such a security unit. That special aspect relates to catheter or cannula arrangements including a sensing element, e.g., a temperature sensing element. In this special aspect, the catheter or cannula arrangement comprises an electronics unit which, e.g., may obviate external reading devices and/or facilitate monitoring the sensed physical quantity such as, e.g., a temperature inside a person's body.

A first aspect relates to the way of determining that it is time for removing the catheter or cannula. It would be possible to determine infection parameters in order to accomplish this. But it turns out that in most cases, it is possible to accomplish this via a measurement of a time duration, more particularly of a preset time span. The preset time span can be, e.g., the longest duration within which, under local and/or personal conditions, no infection is to be expected. One way of implementing the first aspect is to provide a timer determining the duration of such a preset time span. Such a timer can be, e.g., an electronic timer or another kind of clock. As an alternative to a measurement of a preset time span or in addition thereto, it may be provided that the (assumed, measured) dwell time, i.e. the time expected to have passed since insertion of the catheter or cannula is indicated, e.g., by a time display such as a time display of a clock, e.g., indicating the time by numbers such as in hours or hours and minutes, e.g., using a liquid crystal display. And/or the time remaining until the end of the preset time span can be displayed in the described way. In addition thereto or alternatively thereto, it can be provided that the dwell time and/or the time remaining until the end of the preset time span is indicated by wirelessly transmitting data indicative of the respective time. An external device such as a reader may then store and/or visually display the dwell time and/or the time remaining until the end of the preset time span.

A second aspect relates to the first aspect in that it relates to the preset time span and concerns ways of determining a point in time suitable for initiating a time duration measurement for the sought purpose. It has in particular been contemplated that a safe way of starting such a time duration measurement could be to do so in reaction to a removal of a needle from the catheter or cannula arrangement. In many cases, a catheter or cannula arrangement comprises, besides a catheter or cannula, also a needle (and more particularly a needle arrangement including a needle), which is removed from the catheter or cannula shortly after inserting it into a body.

In one embodiment, such an extraction of the needle from the catheter or cannula is used as a starting point for a time measurement. Generally, it would also be possible to start the time measurement in another way, e.g., in reaction to a button being pressed or to a removing of the catheter or cannula arrangement from its package or wrapper, or still otherwise. In another embodiment, in which the catheter or cannula comprises a valve device, the removal of an item, such as a closure or, more generally, an access inhibitor, from the valve device can be used as a starting point for a time measurement, as will be described in more detail below.

Detecting such an event (initiating event or first event) may be accomplished in various ways, e.g., mechanically, electrically (via a change of an electric resistance, such as via an opening or a closing of an electrical connection), magnetically, inductively (e.g,. via a change of an inductivity), optically, optoelectrically. In one embodiment, this is accomplished electrically and more particularly by detecting an opening or a closing of an electric circuit taking place in reaction to the event. It is in particular possible to provide that one or more electrical contact elements are directly or indirectly fixed (e.g., permanently or separably fixed) to the catheter or cannula, whereas another one or more electrical contact elements are fixed elsewhere. The latter may be fixed to the needle or needle arrangement (or to the package or wrapper, or to the item) and thus be (initially) in a defined initial position relative to the catheter or cannula, but removed from this initial position when removing the needle or needle arrangement from the catheter or cannula after insertion of the catheter or cannula (or when opening the package or wrapper, or when removing or unplugging the item). Thus, an electrical contact would be opened, e.g., when extracting the needle out of the catheter or cannula (or in the other cases). On the other hand, one can also use an element which electrically separates contacts (such as spring-loaded contacts) in a defined initial position (with the needle inserted in the catheter or cannula, or with the package or wrapper closed, or with the item in place). On removal of such an element, e.g., by removing the needle or the item from the catheter or cannula, the contacts engage and thus close, which is detectable and may be used as a signal for starting the time measurement.

A third aspect relates to ways of indicating that the time has come to remove the catheter or cannula from the body. In the above-mentioned example (cf. the first aspect above), this would be an indication that the end of the preset time span is reached. Many ways of accomplishing this are possible, e.g., a signal could be emitted by the security unit, such as an optical signal, an acoustic signal, an electric signal, a magnetic signal, an electromagnetic signal, a mechanical signal, a vibration or a vibration signal. It is possible to produce a human-perceivable signal, e.g., in particular an acoustic or an optical signal. But it is also possible to produce a signal for an external unit such as a reader, e.g., an electromagnetic signal in the optical, radio frequency or microwave range, wherein a reader receiving such a signal could then by itself produce a human-perceivable signal in reaction thereto, or transmit a further signal to another apparatus for finally producing a human-perceivable signal.

In particular if emitted by the security unit itself, an optical signal may be produced using one or more light emitting diodes, in particular having a color in the range of red via orange to yellow. Furthermore, the optical signal may be intensity modulated, e.g., include light pulses and more particularly a sequence of light pulses.

As has been mentioned above, alternatively or additionally, the dwell time itself (or the time remaining until the end of the preset time span) may be indicated by, e.g., presenting the respective number of hours, e.g., in a time display, or by wireless transmission of data indicative of the respective time.

A fourth aspect concerns the location or arrangement of the security unit with respect to the catheter or cannula (or to other constituents of the catheter or cannula arrangement). It is generally possible to provide that for some purpose, a device or an apparatus is used which is meant to be distant from and/or not included included in and/or not assigned to the catheter or cannula arrangement, e.g., a reader for use with a multitude of security units for reading a status from a respective security unit or for initiating a time duration determination like the one mentioned above, or a (mobile) charging device for providing electrical energy to an electronic circuit of the security unit, e.g., via induction.

In one embodiment, however, the security unit is completely included in the catheter or cannula arrangement. This way, the security unit may function fully independently of additional infrastructure, making it simple to implement and use.

In particular, it can be provided that one portion of the security unit is fixed to the catheter or cannula, whereas another portion of the security unit is fixed to a needle or needle arrangement of the catheter or cannula arrangement (cf. also the second aspect above).

One way of integrating at least a portion of the security unit in the catheter or cannula arrangement is to have at least a portion of the security unit included included in a holder (or shaft part) holding the catheter or cannula, e.g., the holder radially encompassing a portion of the catheter or cannula and/or forming a portion of the catheter or cannula arrangement staying attached to the catheter or cannula while the catheter or cannula is inserted in the body, but itself remaining (at least predominantly) outside the body, yet possibly in contact with the body.

Another way of integrating at least a portion of the security unit in the catheter or cannula arrangement is to have at least a portion of the security unit included included in an extension of the catheter or cannula arrangement or to have it attached to such an extension.

Such an extension is a part connectable to a connection end of the catheter or cannula arrangement. A catheter or cannula arrangement has a connection end usually at least for the purpose of connecting a device by which a fluid is supplied or removed from the body. The fluid is usually a liquid flowing also through the catheter or cannula. The device may be a tube or a syringe; and often, also a closure, such as a cap, is provided for closing the connection end. Such a connection end is usually present at a holder (or shaft part) holding the catheter or cannula, wherein the holder may be a part distinct from the catheter or cannula or may provide a single integrally formed part together with the catheter or cannula. Accordingly, the extension can more specifically also be understood as an extension of such a holder or shaft part. Typically a needle of the catheter or cannula arrangement, when present, extends through the connection end before the needle is removed out of the catheter or cannula, and with the extension connected, it also extends through the extension. The extension may in particular be adapted to be plugged into the connection end.

The extension may in particular be a part connected, at a first end (which may be referred to as inner end), directly to the connection end; and its second end (which may be referred to as outer end) may reproduce the functionality (more particularly the interconnection functionality) of the connection end.

E.g., with a needle present inside the catheter or cannula, a needle arrangement of the catheter or cannula arrangement and the catheter or cannula (or more particularly a holder or shaft part) are mutually interconnected by the extension.

The extension may, e.g., include a single-piece, e.g., injection-molded, part providing at least a portion of a housing for at least a portion of the security unit.

It can be an elegant way of retrofitting standard catheter or cannula arrangements to provide that at least a portion of the security unit is included in an extension of the catheter or cannula arrangement, in particular in the case where no injection port is present.

Still another way of integrating at least a portion of the security unit in the catheter or cannula arrangement originates in the case of catheters having a valve such as is the case for, e.g., Foley catheters. It is then possible to have at least a portion of the security unit connected to a valve device including the valve. E.g., the catheter or cannula arrangement may include an attachment to the valve device which provides a housing for at least a portion of the security unit. In addition, an item such as a closure for an access end of the valve device can be provided which is operationally connected to that portion of the security unit which is present in the housing. Such an item may provide a portion of an activation detection unit of the security unit.

Another way of integrating at least a portion of the security unit in the catheter or cannula arrangement is to have at least a portion of the security unit included in a cap of the catheter or cannula arrangement or to have it attached to such a cap. Many catheter or cannula arrangements have an injection port, e.g., for dosing medicaments, which can be closed by a removable cap, the cap usually being fastened to the port (or at least to a portion of the catheter or cannula arrangement fixed to the catheter or cannula).

Having at least a portion of the security unit included in such a cap is an elegant and particularly compact way of realizing the fourth aspect. This way, an integrated catheter or cannula arrangement may be realized in the sense that it is a catheter or cannula arrangement in the design of which the security unit has already been considered.

On the other hand, the fourth aspect may be realized in yet another way, namely by having at least a portion of the security unit included in an attachment to such a cap. This way, a retrofit catheter or cannula arrangement may be realized in the sense that an already existing catheter or cannula arrangement (or a corresponding design thereof) may be relied upon while merely adding an attachment to the cap.

If an additional portion of the security unit is provided such as the one attached to the needle or needle arrangement mentioned above (cf. the second aspect), it is also here possible to provide an integrated solution as well as a retrofit solution. If, e.g., an electrical contact element is to be provided attached to the needle or needle arrangement, this may be either integrated in the needle arrangement, e.g., in a handle (also referred to as catheter upstand) of the needle arrangement, or may be included in an attachment thereto. The latter case again is adaptable to pre-existing catheter or cannula arrangements, the former possibly allowing a more compact or more elegant design due to the possibility of considering the electrical contact element in the handle design from the start.

Note that it is not only possible to combine merely retrofit solutions for different portions of the catheter or cannula arrangement or merely integrated solutions for different portions of the catheter or cannula arrangement, but it is also possible to combine a retrofit solution for one portion of the catheter or cannula arrangement, e.g., for a portion included in an attachment to a cap, with an integrated solution for another portion, e.g., for a portion integrated in the needle arrangement.

A fifth aspect relates to a cap of (or for) a catheter or cannula arrangement and to an attachment to such a cap, respectively. In another view, it relates to a housing (or casing) for a security unit or for a portion of a security unit. In case of a cap forming such a housing, one may realize an integrated solution at least similar to the one described above for the fourth aspect. In case of an attachment to a cap forming such a housing, one may realize a retrofit solution at least similar to the one described above for the fourth aspect.

More particularly, in the integrated solution, the cap may be included in a member including including a first part or cover part by means of which a portion of the security unit (such as a printed circuit board assembly) is (partially) housed and/or which at least contributes to holding the portion of the security unit, a second part or plug part or intermediate part by means of which an injection port of the catheter or cannula arrangement can be plugged and/or by means of which a portion of the security unit (such as a printed circuit board assembly) is (partially) housed and/or which at least contributes to holding the portion of the security unit, and a third part or connector part such as a loop for fastening the cap to another portion of the catheter or cannula arrangement, such as to an injection port. More particularly, the first, second and third parts are embodied in a single component, more particularly in a component being one integrally formed part, e.g., in one injection-molded part. And the member may include two interconnecting parts, one interconnecting the first and the second part, the other interconnecting the second and the third part, in particular wherein the interconnecting parts are foldable. And it may be provided that the first and second parts have a snap fit connection (thus forming a snap fit) and/or that the second and third parts have a snap fit connection (thus forming a snap fit).

In the retrofit solution, the attachment to the cap may include a first part or cover part by means of which a portion of the security unit (such as a printed circuit board assembly) is (partially) housed and/or which at least contributes to holding the portion of the security unit, and a second part or snap-on part for attaching the attachment to a cap of a catheter or cannula arrangement and/or by means of which a portion of the security unit (such as a printed circuit board assembly) is (partially) housed and/or which at least contributes to holding the portion of the security unit. More particularly, the first and second parts may be embodied in a single part, more particularly in one integrally formed part, e.g., in one injection-molded part. And the member forming the attachment may include an interconnecting part interconnecting the first and the second part, in particular wherein the interconnecting part is foldable. And it may be provided that the first and second parts have a snap fit connection.

Note that, in any event, a retrofit solution (realized in any of the described ways) may be looked upon and treated and realized separately from an integrated solution (realized in any of the described ways).

A sixth aspect relates to an extension of (or for) a catheter or cannula arrangement. A typical kind of extension has been described elsewhere in the present patent application, e.g., above, in conjunction with the fourth aspect. In another view, the sixth aspect relates to a housing (or casing) for a security unit or for a portion of a security unit. When the extension includes or provides such a housing, one may realize a solution at least similar to the one described above for the fourth aspect. The provision of the extension may make possible to realize a retrofit solution for known (standard) catheter or cannula arrangements, cf. also the fourth aspect above.

The extension may, e.g., include and/or provide a first part which is a housing part by means of which at least a portion of the security unit (such as a printed circuit board assembly) is fully or partially housed. The extension may, in addition to the first part, include a second part connected to or connectable to the first part, such that they enclose an inner volume to accommodate the portion of the security unit. More particularly, the first part and the second part may, e.g., each be embodied as a single component, more particularly as a component being one single integrally formed part, e.g., one injection-molded part, or, e.g., both, the first part and the second part, may be embodied in one single component, more particularly in a component being one integrally formed part, e.g., in one injection-molded part. In case the first part and the second part are distinct components, but also if they are integrally formed, they may, e.g., have a snap fit connection.

The extension may be included in (and thus may itself be) a device having the following properties: A device including an extension connectable, in particular fastenable, at its first end, to a connection end of a catheter or cannula arrangement, the extension providing an inner channel for flow of a fluid through the catheter or cannula. And, in particular, at its second end, the extension may provide a connector for connecting, in particular fastening, a device by which the fluid is supplied or removed and/or may reproduce the connectivity of the connection end. Furthermore, the extension provides an inner volume separate from the inner channel for at least partially housing and/or at least contributing to locating a printed circuit board (or possibly of another portion of a security unit for a catheter or cannula arrangement).

For each of the aspects described above, one or more possible solutions and embodiments, respectively, have been contemplated which are described above or elsewhere in the present patent application. These (partial) embodiments and solutions, respectively, may be combined in any logically possible way.

And certainly, the invention may include not only catheter or cannula arrangements, but also security units and security unit arrangements and caps and attachments to caps and needle arrangements (for use with a catheter or cannula or for use in a catheter or cannula arrangement) as well as methods for monitoring a length of stay of the catheter or cannula in a body, and manufacturing methods.

And the invention further includes electronics unit arrangements and methods for monitoring a physical quantity sensed by a sensing element of a catheter or cannula arrangement.

One object of the invention is to create an improved security in the use of catheter or cannula arrangements.

Another object of the invention is to provide improved catheter or cannula arrangements.

Another object of the invention is to provide catheter or cannula arrangements which can be operated safely.

Another object of the invention is to do so in a particular cost-efficient way and/or in a particularly reliable way and/or in a particularly robust way and/or in a particularly easily manufacturable way.

Further objects emerge from the description and embodiments above or below.

At least one of these objects is at least partially achieved by apparatuses and methods according to the patent claims.

In the following, several embodiments are discussed more specifically.

The catheter or cannula arrangement includes
a catheter or cannula;
a security unit for monitoring a length of stay of the catheter or cannula in a body.

In the before-mentioned special aspect, the provision of the security unit is optional.

The security unit may prevent the catheter or cannula staying in a body for too long.

It may in particular be provided that at least a portion of the security unit is indirectly attached to the catheter or cannula.

In one embodiment, the arrangement comprises an injection port and a cap for sealing the injection port, wherein a portion of the security unit is arranged
i) in the cap; or
ii) in an attachment to the cap.

Case i) can contribute to realizing an integrated solution, whereas case ii) can contribute to realizing a retrofit solution (cf. the fifth aspect above).

In particular, the cap and the attachment to the cap may form a housing for at least a portion of the security unit.

In one embodiment referring to the last-mentioned embodiment, the cap includes an integrally formed member including, in case i),
a first part for at least partially housing and/or at least contributing to locating a portion of the security unit, wherein for the purpose of the locating, one or more protrusions may be provided in the first part for engagement with the portion of the security unit;
a second part attached to or attachable to the first part (in particular by means of a snap fit), for plugging the injection port and/or for at least partially housing and/or at least contributing to locating a portion of the security unit, wherein for the purpose of locating, one or more protrusions may be provided in the second part for engagement with the portion of the security unit; and
a third part attached to or attachable to the second part (in particular by means of a snap fit), for fastening the cap to another portion of the catheter or cannula arrangement such as to the injection port;
   in case ii),
a first part for at least partially housing and/or at least contributing to locating a portion of the security unit, wherein for the purpose of the locating, one or more protrusions may be provided in the first part for engagement with the portion of the security unit; and
a second part attached to or attachable to the first part (in particular by means of a snap fit), for attaching the attachment to the cap (in particular by means of a snap fit) and/or for at least partially housing and/or at least contributing to locating a portion of the security unit, wherein for the purpose of locating, one or more protrusions may be provided in the second part for engagement with the portion of the security unit.

In case ii), the second part may in particular include one or rather two or more portions for accomplishing, in cooperation with at least one edge of the cap, a snap fit with the cap.

If the first and second parts (in case i) or case ii)) are to hold a portion of the security unit such as, e.g., a printed circuit board assembly, they may include protruding portions for mechanically positioning that portion from two substantially opposite sides, e.g., by engaging with a printed circuit board of the printed circuit board assembly from both (i.e. upper and lower) sides.

In one embodiment which may be combined with one or more of the before-mentioned embodiments, the arrangement includes a connection end and an extension connected to the connection end, wherein at least a portion of the security unit is included in or attached to the extension. Such an extension can make possible retrofitting catheters with a security unit. The extension may provide a housing for at least a portion of the security unit. E.g., the extension is a part adapted to be plugged to the connection end (at which a tube or a syringe or other devices can be connected to the catheter or cannula arrangement) which reproduces the connectivity of the connection end.

The extension may include or in particular substantially consist of a generally tubular part and a housing part, the tubular part providing an inner channel and two connectors, the housing part providing the housing for at least a portion of the security unit. The housing part is connected to the generally tubular part typically at the periphery of the generally tubular part.

In one embodiment which may be combined with one or more of the before-mentioned embodiments, the arrangement comprises a valve device, wherein at least a portion of the security unit is connected to the valve device. The valve device includes a valve and usually is present at an outer end of the catheter or cannula arrangement (which remains outside the body). The inner end of a catheter or cannula is the end to be present in the body.

At least a portion of the security unit can in particular be fastened to the valve device.

It is possible to provide that the security unit (in full) is connected, in particular fastened, to the valve device.

The arrangement may include a first channel and a second channel which are separate from each other, wherein the valve device is present at an end of the second channel. Foley catheters have such two channels.

The catheter or cannula arrangement may include a Foley catheter.

It is possible to provide that at the inner end of the catheter or cannula, a balloon is present which is inflatable via the second channel. The valve device (and more particularly the valve) makes possible to enable or inhibit an exchange of a fluid between the second channel and the exterior.

In many cases, arrangements including a valve device do not include, in addition, a needle and/or an extension and/or an injection port.

In one embodiment referring to the before-mentioned embodiment, the arrangement includes an attachment to the valve device, the attachment constituting a part of the security unit, in particular wherein at least a portion of the security unit is located in the attachment to the valve device.

The attachment to the valve device may, e.g., form a snap fit with the valve device.

The attachment to the valve device may, e.g., surround or radially encompass the valve device at least in part.

The attachment to the valve device may however also be, e.g., attached to an access end of the valve device wherein the attachment may, e.g., be a closure or cover or another item.

In one embodiment referring to the before-mentioned embodiment, the valve device has an access end, and the security unit includes an item, e.g., a closure, attached to the access end operationally connected to the portion of the security unit located in the attachment to the valve device.

The access end typically is that end of the valve device at which a fluid may be input into the second channel and/or at which a fluid can leave the second channel.

The item is provided for inhibiting access to the access end and may thus be considered an access inhibitor.

The operational connection can in particular be provided by a separating element, in particular by a strip-shaped separating element. But other embodiments are possible, cf., e.g., the before-described second aspect.

In one embodiment which may be combined with one or more of the before-mentioned embodiments, the security unit includes a signalling unit for at least one of:
emitting a first signal indicative of an attainment of an end of a preset time span;
indicating a time corresponding to the length of stay or corresponding to a time remaining up to a preset time span.

The preset time span can in particular be between 2 days and 6 days, more particularly between 60 hours and 120 hours, e.g., between 72 hours and 96 hours. This can contribute to effectively prevent problems such as infections due to the catheter or cannula remaining too long in the body. For specific catheters or cannulas, the preset time span may, however be notably different therefrom, e.g., for urinary catheters, e.g., Foley catheters, it will typically rather be between 7 days and 120 days, e.g., up to 90 days, or in particular between 14 days and 60 days, e.g., between 21 days and 40 days.

It is also possible to provide a signalling unit for indicating the length of stay (dwell time) itself, or the time remaining until the preset time span will elapse. In this case, the signalling unit may be or at least include, e.g., a clock indicating hours, or hours and minutes. The signalling unit can, in this case, indicate the respective time for instance visually e.g., in a way an ordinary clock does such as using a numeric display.

In one embodiment referring to the last-mentioned embodiment, the first signal is at least one of
a visual signal;
an optical signal;
an acoustic signal;
an electric signal;
a magnetic signal;
an electromagnetic signal, in particular an electromagnetic signal in the optical, the radio frequency or the microwave range;
a mechanical signal;
a vibration.

In case the first signal includes a visual, in particular an optical signal, the signalling unit may include, e.g., a clock display as mentioned above, or at least one light emitter such as at least one light emitting diode (LED). It may be provided that the signalling unit is capable of emitting light of at least two different colors. This may be accomplished using one suitable light emitter or rather at least two light emitters for emitting light of different colors such as red and green light, e.g., two corresponding LEDs. An arrangement of light emitters may be provided in the cap or in the attachment to the cap. The cap or the attachment to the cap may, in this case, include, for letting escape the light from the cap or from the attachment to the cap, one or more openings or one or more transparent portions in the cap and the attachment, respectively; e.g., one such opening or transparent portion may be provided for each light emitter. In case an arrangement of light emitters is provided elsewhere (not in a cap or in an attachment to the cap), but in another housing, the described features can be provided by the housing, e.g., by a housing provided by an extension or by a housing provided by an attachment to a valve device. The signalling unit may be provided for emitting light pulses, in particular during the before-mentioned preset time span. And it may be provided that after a preset time span, it emits other light pulses and/or a different sequence of pulses.

If a human-perceivable signal is not or not only provided by the signalling unit itself (or at least not by a portion of the signalling unit attached to the catheter or cannula) but by a remote device or remote apparatus, electromagnetic signals, e.g., in the radio frequency range (or in the optical range, or in the microwave range) can be particularly useful. Remote alerts can be realized this way. In case of optical signals, light pulses may be emitted encoding time information indicating, e.g., the dwell time or the time remaining until a preset time span will have elapsed. Such optically encoded time information may be read by a reader, e.g., the reader including one or more photo detectors such as photodiodes. And the reader may include a visual display or be operationally connected to a visual display, for visually indicating the transmitted time.

In one embodiment which may be combined with one or more of the before-mentioned embodiments, the security unit includes an activation detection unit for detecting a first event and for producing a second signal indicative of the detection. In particular, the first event can be at least one of
a relative movement of a portion of the arrangement and another portion of the arrangement, in particular wherein the catheter or cannula arrangement includes a needle and wherein the relative movement is a pulling-out movement by means of which the needle is moved at least partially out of the catheter or cannula;
a removal of a portion of the arrangement from another portion of the arrangement, such as, e.g., a removal of a needle from the catheter or cannula;
an application of a force to at least a portion of the arrangement such as, e.g., pressing a button, squeezing a portion of the catheter or cannula arrangement, undoing a connection of two parts of the catheter or cannula arrangement;
a deformation of at least a portion of the arrangement such as, e.g., pressing a button, squeezing a portion of the catheter or cannula arrangement.

In case of the relative movement or of the removal of a portion of the arrangement from another portion of the arrangement, the one portion may include the catheter or cannula, and the other portion may include the needle insertable into the catheter or cannula or inserted in and extractable from the catheter or cannula. In a common procedure of using a catheter or cannula, a needle is pulled out of the catheter or cannula. Using this step or a consequence thereof as the first event makes it possible to dispense with extra action. In other words, personel such as nurses handling catheters or cannulas will “automatically” cause the first event when using them in the usual way. Accordingly, healthcare workers dealing with catheters or cannulas do not need to carry out any additional action, spend any more time or change their working technique in any way. Thus, neither working practice nor standard of care are compromised while ensuring that the described security measures can be accomplished, in particular, a dwell time measurement can be started.

Instead of involving a movement or removal of a needle for detecting the first event, it is also possible to make use of a movement or removal of an item attached to an access end of a valve device for this purpose. In case of catheter or cannula arrangements with valve devices, the time of applying (introducing) the catheter or cannula approximately coincides with the time of using the valve, e.g., for inflating a balloon of the catheter or cannula arrangement. Accordingly, a detection of a removal of an item inhibiting access to the access end of the valve device is a good indication for the beginning of the dwell time.

In one embodiment which may be combined with one or more of the before-mentioned embodiments, the security unit includes a timer unit for determining a length of a time span.

In one embodiment referring to the last-mentioned embodiment, but with the before-mentioned signalling unit and activation detection unit provided, the timer unit is operationally connected to the activation detection unit and to the signalling unit, for receiving the second signal and for starting a time duration measurement in reaction to the receiving the second signal and in particular for producing and transmitting to the signalling unit a third signal as soon as the measured time duration is as large as or exceeds the preset time span, and wherein the signalling unit is structured and configured for emitting the first signal in reaction to receiving the third signal. In particular, it may be provided that the time duration measurement is started immediately upon reception of the second signal. The production and transmission of the third signal may be dispensed with, e.g., in case the signalling unit displays a time, such as the time (dwell time) elapsed since occurrence of the event or the time remaining until a preset time span having started at the occurrence of the event will have elapsed. The signalling unit may alternatively or additionally be structured and configured for indicating, e.g., displaying, the measured time duration or the time remaining until the preset time span is reached.

In one embodiment which may be combined with one or more of the before-mentioned embodiments, the security unit comprises an integrated circuit. That integrated circuit may include a portion of the signalling unit, a portion of the activation detection unit, and at least a portion of the timer unit. In case of the before-mentioned special aspect, a portion of the electronics unit may be included in the integrated circuit.

In one embodiment which may be combined with one or more of the before-mentioned embodiments with the timer unit and the activation detection unit, the timer unit is structured and configured for starting the time duration measurement only after a preset delay time has elapsed after the receiving the second signal, in particular wherein the preset delay time is between 1 and 30 seconds, more particularly between 3 and 20 seconds, even more particularly between 6 and 15 seconds. This may be helpful for preventing an undesired start of the time duration measurement which might occur, e.g., by an accidental removal of a needle from the catheter or cannula for a short time or for test reasons.

In one embodiment referring to the last-mentioned embodiment, the timer unit is structured and configured for receiving a fourth signal and for starting the time duration measurement only if the fourth signal is not received in the timer unit before expiry of the preset delay time.

In one embodiment referring to the last-mentioned embodiment, the activation detection unit is structured and configured for producing the fourth signal.

In one embodiment referring to the last-mentioned embodiment, the activation detection unit is structured and configured for detecting a second event, the fourth signal being indicative of a detection of the second event.

In one embodiment referring to the last-mentioned embodiment, the second event is inverse to the first event.

For example, if the catheter or cannula arrangement comprises a needle insertable into the catheter or cannula or inserted in and extractable from the catheter or cannula, and if the first event is a removal movement by which the needle is at least partially removed from the catheter or cannula, the second event may be a re-insertion movement by which the needle is inserted into or at least inserted further into the catheter or cannula.

In one embodiment which may be combined with one or more of the before-mentioned embodiments, provided that the first event is an at least partial extraction of a needle from the catheter or cannula, the activation detection unit is structured and configured for detecting the at least partial extraction of the needle from the catheter or cannula in at least one of the following ways:
mechanically;
electrically;
via a change of electric resistance;
inductively;
capacitively;
magnetically;
via a change of inductivity;
optically.

Detecting the first event electrically and, more specifically, via a change of an electric resistance turns out to be very suitable for a desired implementation, in particular when an opening or a closing of an electrical circuit or contact is detected. For example, an electrical contact closing an electrical circuit in an initial position (needle inserted in catheter or cannula) could be opened by removing one or more electrical contact elements from further one or more electrical contact elements by pulling out the needle, or an electrical contact which is, in an initial position, open because of an electrically insulating element being located between two electrical contacts, could be closed by removing the electrically insulating element by pulling out the needle. Instead of involving an at least partial extraction of a needle for detecting the first event, it is also possible to make use of a movement or removal of an item (access inhibitor) attached to an access end of a valve device for this purpose. Accordingly, the detection of the first event may also in this case be accomplished in one or more of the above-described ways.

In one embodiment referring to the last-mentioned embodiment, the activation detection unit comprises a first portion directly or indirectly fixed to the catheter or cannula and a second portion directly or indirectly fixed to the needle or to the item attached to an access end of a valve device.

In one embodiment referring to the last-mentioned embodiment, the catheter or cannula arrangement comprises a needle arrangement including the needle, wherein the second portion of the activation detection unit
I) is integrated or included in the needle arrangement; or
II) is integrated or comprises an attachment to the needle arrangement.

Case I) may contribute to realizing an integrated solution. Case II) may contribute to realizing a retrofit solution.

The attachment to the needle arrangement may, e.g., be mechanically attached to needle arrangement, be slid on needle arrangement, form a snap fit with needle arrangement, be glued to needle arrangement or be otherwise fixed thereto.

In one embodiment referring to the last-mentioned embodiment, the needle arrangement includes a handle, and, in case I), the second portion of the activation detection unit is integrated or included in the handle and, in case II), the second portion of the activation detection unit is integrated or included in the attachment to the handle. The handle may be provided for facilitating extracting the needle from the catheter or cannula.

In one embodiment referring to the last-mentioned embodiment, in case II), the attachment includes a channel or groove slidable onto the handle, the attachment thus being attachable to the handle by the channel or groove.

In one embodiment referring to the last-mentioned embodiment, in case II), a first of the handle and the attachment to the handle has a recess or opening and a second of the handle and the attachment to the handle has a protrusion for cooperating with the recess or opening. This may be provided for defining and/or stabilizing a position of the attachment to the handle while being attached to the handle. The same applies completely analogously where the attachment is attached to a portion of the needle arrangement different from a handle.

In one embodiment referring to one or more of the three last-mentioned embodiments, the second portion of the activation detection unit includes at least one of
an electrical contact element; and
an element for electrically separating electrical contact elements, referred to as separating element.

Therein, it can in particular be provided that the electrical contact element and the separating element, respectively, are operational and/or functional parts of the activation detection unit.

The electrical contact element may, in particular, provide that an electric circuit of the security unit is closed when the needle is inserted in the cathether or cannula and is open when the needle is removed from the cathether or cannula. And the separating element may, in particular, provide that two electrical contact elements of the security unit are electrically separated from each other when the needle is inserted in the catheter or cannula (initial position) and that these two electrical contact elements are in electrical, in particular galvanic, contact with each other when the needle is removed from the cathether or cannula (removed together with the needle or needle arrangement). The separating element may, e.g., be (at least substantially) made of an electrically insulating material or, at least, include at least a portion which is (at least substantially) made of an electrically insulating material (e.g., a polymer), and the two electrical contact elements may, e.g., form a spring-loaded contact (which is separable by the separating element when it is arranged between the two electrical contact elements). The separating element may exhibit the shape of a sheet or strip or of a tongue, and it may be a sheet or strip or a tongue. It may substantially be or at least include a foil, in particular a polymer foil.

It is noted that the embodiments with the electrical contact element and/or with the separating element can also be realized in the case where an at least partial removal of an item attached to an access end of a valve device is used as a first event instead of the at least partial extraction of a needle described in great detail. Changes to be applied in that case (with respect to the needle-removal case) are obvious. This applies also for the embodiments further below.

In one embodiment referring to the last-mentioned embodiment, the second portion of the activation detection unit includes a contacting portion in which the electrical contact element is arranged, the electrical contact element being, in case I), integrated in or fixed to the needle arrangement and, in case II), integrated in or fixed to the attachment to the needle arrangement, in particular wherein the electrical contact element is included in the second portion of the activation detection unit.

Therein, the contacting portion may form a protruding portion. And it may, in particular, be provided that the electrical contact element is,
in case I), integrated in or fixed to a handle of the needle arrangement, and,
in case II), integrated in or fixed to an attachment to a handle of the needle arrangement.

In one embodiment which may be combined with one or more of the before-mentioned embodiments in which the activation detection unit includes the first and second portions, the first and second portions of the activation detection unit each include at least one electrical contact element. In particular, it may be provided that the cap or the attachment to the cap has a protruding portion in which at least one electrical contact element of the first portion of the activation detection unit is arranged.

In one embodiment referring to the last-mentioned embodiment, the electrical contact elements are structured and arranged such that they form a closed electrical contact when the needle is in an inserted position in the catheter or cannula and that they are separated from each other when the needle is extracted from the catheter or cannula. Therein, it may be provided that the closed electrical contact is a slidable electrical contact and/or that the closed electrical contact is a clamping electrical contact. For example, the closed electrical contact may include a resilient contact piece mechanically pressing from two opposing sides onto a part (such as a printed circuit board) including another contact piece formed by, e.g., contact leads of a printed circuit board.

In one embodiment which may be combined with one or more of the before-mentioned embodiments in which the activation detection unit includes the first and second portions, the first portion of the activation detection unit includes a first and a second electrical contact element, the second portion of the activation detection unit includes an electrical contact element referred to as third electrical contact element, and wherein the first, second and third electrical contact elements are structured and arranged such that
the third electrical contact element bridges the first and second electrical contact elements when the needle is inserted in the catheter or cannula; and
the third electrical contact element does not bridge the first and second electrical contact elements when the needle is extracted from the catheter or cannula.

Therein, the status or situation “when the needle is inserted in the catheter or cannula” may in particular correspond to the earlier mentioned defined initial position.

In one embodiment referring to the last-mentioned embodiment, at least one of the following applies:
the first and second contact elements are formed by contact leads or tracks of a printed circuit board; and
the third contact element is formed by a piece of sheet metal, more particularly by a clamp or clamping element formed by sheet metal.

Other electrical contact elements are possible, too, such as contact pins and a sheet metal strip or a metallic coating.

The piece of sheet metal may be, in particular, a bent piece of sheet metal forming a clamp or jaws with a tapering entrance for facilitating an introduction of the first and second contact elements (or a part including these). Alternatively, it may be a sheet metal tongue for bridging the first and second contact elements.

In one embodiment which may be combined with one or more of the before-mentioned embodiments in which the activation detection unit comprises the separating element, the separating element includes an electrically insulating portion, in particular, wherein the electrically insulating portion is made of electrically insulating material, and more particularly is formed by an electrically insulating foil or an electrically insulating member having a sheet shape.

More particularly, the separating element may substantially be the electrically insulating portion, i.e. it may be made of electrically insulating material. Furthermore, the separating element or the electrically insulating portion may be, e.g., a pull-out tab or strip.

In one embodiment referring to the last-mentioned embodiment, the separating element substantially is a foil or sheet of an electrically insulating polymer.

In one embodiment referring to one or both of the two last-mentioned embodiments, the security unit comprises a first and a second electrical contact element, wherein the separating element and the first and second electrical contact elements are structured and arranged such that
when the needle is inserted in the catheter or cannula, the electrically insulating portion is arranged in a location between the first and second electrical contact elements, electrically separating the first from the second contact element; and
when the needle is extracted from the catheter or cannula, the electrically insulating portion is removed from the location allowing the first and second contact elements to be in electrical contact with each other.

The situation “when the needle is inserted in the catheter or cannula” may in particular correspond to the earlier mentioned defined initial position. And, the insulating portion may in particular be clamped between the first and second electrical contact elements. Furthermore, the first and second electrical contact elements may in particular form a closed spring-loaded electrical contact when the electrically insulating portion is removed from between them. For example, the first electrical contact element may be a piece of sheet metal and/or the second electrical contact element may be a contact pad of a printed circuit board.

It may be provided that, when removing the needle from the catheter or cannula, the insulating portion is removed from the location in which it separates the first and second contact elements from each other.

In one embodiment referring to one or both of the three last-mentioned embodiments, the separating element has a first end and a second end, the electrically insulating portion being located at its first end, and the separating element being fixed at its second end, in case I), to the needle arrangement and, in case II), to the attachment to the needle arrangement.

More specifically, the separating element can be fixed at its second end, to a handle of the needle arrangement or to an attachment to such a handle. The following paragraphs will simply refer to the handle (and to the attachment thereto), but more generally could instead apply to another portion of the needle arrangement, e.g., to a holder or shaft part holding the needle.

The separating element can be mechanically fixed to the handle or attachment to the handle, or it can be fixed thereto by gluing or by fusing (locally melting the separating element and/or the handle or attachment to the handle, and bringing the parts in contact) or otherwise. In particular, the separating element and/or the handle or the attachment to the handle may have one or more means for providing a mechanical connection between the second end of the separating element and the handle or the attachment to the handle. For example, it can be provided that the separating element has at least one hole, in particular at least one through-hole, and the handle or the attachment to the handle has at least one protrusion extending through the at least one hole. Therein, the at least one protrusion may have a waist dimensioned so as to accommodate the at least one hole but impeding a removal of the separating element from the handle or the attachment to the handle.

It is possible to consider a printed circuit board assembly of the security unit (which will be described in more detail below) provided with the separating element and the attachment to the handle to be a separate assembly. Such an assembly is manufacturable separately from further parts of the catheter or cannula arrangement. For example, in the assembly, the first end of the separating element is located such that it separates electrical contact elements of the printed circuit board assembly; and the second end is fixed to the attachment to the handle. And, e.g., during assembly of the catheter or cannula arrangement, the printed circuit board assembly is attached or fixed to the portion of the catheter or cannula arrangement including the catheter or cannula, i.e. to that portion of the catheter or cannula arrangement which will remain at the body when the catheter or cannula is used, and the attachment to the handle is fixed to the handle (while the needle is staying in the catheter or cannula). As has been pointed out before, although only the handle has been referred to before, the same may apply as well to another portion of the needle arrangement. The assembly will be described below in greater detail.

In one embodiment which may be combined with one or more of the before-mentioned embodiments, the security unit includes a printed circuit board assembly including a printed circuit board. Therein, it may be provided that at least a portion of the printed circuit board assembly is molded or is covered by a conformal coating, in particular for protecting the printed circuit board assembly or portions thereof. A battery of the security unit may be inside or outside the conformal coating.

The printed circuit board assembly may in particular include at least a portion of the signalling unit, more particularly the signalling unit in full, a portion of the activation detection unit, at least a portion of the timer unit, more particularly the timer unit in full.

And, electrical contact(s) of the activation detection unit (more particularly of the first portion of the activation detection unit) may be included in the printed circuit board assembly.

Where at least a portion of the security unit is included in or attached to a cap for closing an injection port of the catheter or cannula arrangement, at least a portion of the printed circuit board assembly may be separated from the catheter or cannula
by a plug for plugging the injection port;
by valve for selectably closing the injection port;
by being (partially) cast, e.g., in an epoxy resin;
by being (partially) covered by a conformal coating.

In particular where the catheter or cannula arrangement does not include an injection port, the printed circuit board arrangement or at least a portion thereof may be included in a holder (or shaft part) holding the catheter or cannula.

In one embodiment which may be combined with one or more of the before-mentioned embodiments, the security unit comprises an energy storage unit. The energy storage unit may in particular be one or more of a battery (in particular a non-rechargeable battery), a capacitor, a rechargeable battery, an inductively chargeable energy storage unit. Where the security unit includes a printed circuit board assembly, the energy storage unit may be mounted on one side of printed circuit board (e.g., by a battery contact or by soldering), and further components of the printed circuit board assembly, such as an integrated circuit (e.g., including timer unit) and LEDs, on the opposite side of the printed circuit board. This can be particularly space-saving.

In one embodiment which may be combined with one of the before-mentioned embodiments, the arrangement includes a sensing element. Such a sensing element usually produces signals related to and in particular indicative of a physical quantity, such as a temperature. The corresponding catheter or cannula can in particular be a urinary catheter or a Foley catheter.

In one embodiment referring to the last-mentioned embodiment, the arrangement includes an electronics unit for processing signals produced by the sensing element and/or for storing data related to signals produced by the sensing element.

In one embodiment referring to the last-mentioned embodiment, at least one of the following applies:
at least a portion of the electronics unit and at least a portion of the security unit are located in one and the same housing;
the electronics unit and the security unit share at least one integrated circuit chip;
the electronics unit and the security unit share a transmitter or transceiver, in particular a radio frequency transmitter or transceiver, for wirelessly transmitting data related to signals produced by the sensing element and for wirelessly transmitting data related to the dwell time;
the electronics unit and the security unit share a common display;
the electronics unit and the security unit share a common energy storage unit, in particular a common battery;
the electronics unit and the security unit share at least one common printed circuit board.

The data related to the dwell time can comprise, e.g., data indicating that the end of the preset time span is reached, data indicative of the dwell time, data indicative of the time remaining until the end of the preset time span.

The two last-mentioned embodiments are embodiments related to the special aspect mentioned further above. They involve the presence of the electronics unit. As has been indicated above, too, the special aspect does not necessarily require the presence of a security unit and may therefore relate to a special embodiment, namely to a catheter or cannula arrangement including
a catheter or cannula;
a sensing element;
an electronics unit for processing signals produced by the sensing element and/or for storing data related to signals produced by the sensing element.

In such a special embodiment, usually at least two channels, in particular one for guiding a fluid (usually a liquid such as predominantly water or urine) and one being an electrical channel (typically wire-bound) are present.

In one embodiment referring to the special aspect, at least a portion of the electronics unit, in particular the electronics unit substantially in full, is arranged in a defined initial position relative to the catheter or cannula, in particular in a fixed position relative to the catheter or cannula.

It may in particular be provided that at least the portion of the electronics unit is present in a housing attached to, in particular fixedly attached to, the catheter or cannula, e.g., to tubing of the catheter or cannula, wherein it may in particular be attached to tubing of each channel of the catheter or cannula arrangement.

In one embodiment referring to one or more of the embodiments related to the special aspect, the arrangement includes at least one of
a storage unit for storing data related to signals produced by the sensing element, e.g., random access memory, flash memory;
electronic circuitry, e.g., realized in an integrated circuit, for processing signals produced by the sensing element and/or for obtaining data related to signals produced by the sensing element;
a display for displaying the or other data related to signals produced by the sensing element;
a user control;
an interface, in particular a wire-bound interface, for outputting the or other data related to signals produced by the sensing element, in particular wherein the interface is a digital interface;
a radio frequency transmitter or transceiver for wirelessly transmitting the or other data related to signals produced by the sensing element.

It is noted that the electronics unit usually includes an energy storage unit, e.g., a battery, and an element for electrically separating electrical contact elements, referred to as separating element, namely for the purpose of having the energy storage unit in an unloaded state with the separating element in an initial position (in which the electrical contact elements are electrically separated by the separating element), and in a loaded state (in which it provides energy to further components of the catheter or cannula arrangement such as the electronic circuitry) with the separating element removed from its initial position (such that the electrical contact elements in mutual electrical contact). More details concerning separating elements are described in conjunction with activation detection units, cf. there.

When an electronics unit is present in combination with a security unit, it can be provided that the electronics unit is (initially) provided with power in reaction the same event as is the security unit and thus simultaneously with the security unit. And/or the activation detection unit of the security unit is, at least in part, also included in and used for the electronics unit. And/or the processing of signals produced by the sensing element and/or the storing data related to signals produced by the sensing element carried out by the electronics unit is initiated in reaction the same event as is the time duration measurement carried out by the security unit. Thus, these will be initiated and usually also start simultaneously.

The invention also includes security units, more specifically a security unit for a catheter or cannula arrangement including a catheter or cannula, which is structured and configured for monitoring a length of stay of the catheter or cannula in a body.

In one embodiment, the security unit includes:
a signalling unit for emitting a signal referred to as first signal;
an activation detection unit for detecting a first event and for producing a signal referred to as second signal indicative of the detection;
a timer unit for receiving the second signal from the activation detection unit and for starting a time duration measurement in reaction to the receiving the second signal;

In particular, the timer unit may be provided for producing and transmitting to the signalling unit a third signal as soon as the measured time duration is as large as or exceeds the preset time span, wherein the signalling unit is structured and configured for emitting the first signal in reaction to receiving the third signal.

As has been explained above, the signalling unit may, alternatively or in addition, display a time, e.g., in numbers.

The invention includes security units with features of corresponding catheter or cannula arrangements according to the invention, and, vice versa, also catheter or cannula arrangements with features of corresponding security units according to the invention.

The advantages of the security units basically correspond to the advantages of corresponding catheter or cannula arrangements, and, vice versa, the advantages of the catheter or cannula arrangements basically correspond to the advantages of corresponding security units.

The invention also includes security unit arrangements, more specifically a security unit arrangement for a catheter or cannula arrangement, which includes a security unit according to the invention.

The security unit arrangement may include a first housing part housing at least a portion of the security unit, in particular wherein the first housing part comprises, and in particular substantially is, an integrally formed member including at least a first and a second part and a foldable interconnecting part interconnecting the first and second parts, the first and second parts in particular forming a snap fit.

The security unit arrangement may alternatively or in addition include a second housing part housing at least a portion of the security unit, in particular wherein the second housing part houses or accommodates at least one electrical contact element referred to as third contact element, wherein the third contact element may be substantially formed by a piece of sheet metal.

The invention includes security unit arrangements with features of corresponding security units or of corresponding catheter or cannula arrangements according to the invention, and, vice versa, also catheter or cannula arrangements or security units with features of corresponding security unit arrangements according to the invention.

The advantages of the security unit arrangements basically correspond to the advantages of corresponding security units or of corresponding catheter or cannula arrangements, and, vice versa, the advantages of the security units or catheter or cannula arrangements basically correspond to the advantages of corresponding security unit arrangements.

The invention also includes assemblies for use with or in a catheter or cannula arrangement, in particular in a catheter or cannula arrangement according to the invention: The assembly includes in particular
a printed circuit board assembly including a first and a second electrical contact element;
a separating element having a first end and, distant therefrom, a second end, the first end being located between the first and second electrical contact elements, electrically separating these from each other, the second end being provided with a fixing element for fixing the second end to another portion of the catheter or cannula arrangement.

In particular, the separating element can substantially be a piece of foil, a piece of sheet shape, or a strip.

In one embodiment of the assembly, the fixing element is a clamping element, more particularly, a clamping element for clamping onto another member of the catheter or cannula arrangement. It can in particular be provided that the clamping element and the other member are complementary parts.

In one embodiment of the assembly which may be combined with the before-addressed embodiment, at least one of the following applies:
the fixing element has an enlargement, and the other member has an opening for accommodating the enlargement;
the other member has an enlargement, and the fixing element has an opening for accommodating the enlargement;
the fixing element and the other member have a snap fit connection.

It may furthermore be provided that the enlargement has a shaft shape or a ridge shape or a knob shape. Fixing the two components to each other may be accomplished, e.g., by sliding the one component onto the other.

In one embodiment of the assembly which may be combined with one or more of the before-addressed embodiments, the separating element has at least one hole, and the fixing element includes at least one protrusion extending through the at least one hole so as to fix the separating element to the fixing element.

It may furthermore be provided that the at least one protrusion has a waist, as has already been discussed above.

The invention includes assemblies with features of corresponding catheter or cannula arrangements or security units according to the invention, and, vice versa, also catheter or cannula arrangements and security units with features of corresponding assemblies according to the invention.

The advantages of the assemblies basically correspond to the advantages of corresponding catheter or cannula arrangements and security units, and, vice versa, the advantages of the catheter or cannula arrangements and security units basically correspond to the advantages of corresponding assemblies.

The invention also includes methods for manufacturing an assembly for use in or with a catheter or cannula arrangement. The method for manufacturing an assembly for use in or with a catheter or cannula arrangement includes the steps of
inserting between a spring loaded pair of electrical contact elements of the assembly a separating element for electrically separating these electrical contact elements from each other;
attaching to the separating element a fixing element for fixing the separating element to another portion of the catheter or cannula arrangement.

The invention includes methods for manufacturing an assembly with features of corresponding assemblies according to the invention, and, vice versa, also assemblies with features of corresponding methods for manufacturing an assembly according to the invention.

The advantages of the methods for manufacturing an assembly basically correspond to the advantages of corresponding assemblies, and, vice versa, the advantages of the assemblies basically correspond to the advantages of corresponding methods for manufacturing an assembly.

The invention also includes needle arrangements, in particular needle arrangements for use with a catheter or cannula or more particularly for use in a catheter or cannula arrangement, and even more specifically for use in a catheter or cannula arrangement according to the invention.

The needle arrangement for use with a catheter or cannula or for use in a catheter or cannula arrangement, in particular in a catheter or cannula arrangement according to the invention includes a needle and a member fixed to the needle, the member including or being provided with at least one of
an electrical contact element;
a protruding portion protruding in a direction along the needle;
an attachment to the member;
a fixing element fixed to the member, in particular for fixing a device or an element to the member;
an electrically insulating strip, in particular a foil or sheet of an electrically insulating polymer;
a holding or clamping element.

Usually, none of these is formed by the needle.

The member may, in particular, be a holder or shaft part holding the needle or a handle fixed to the needle.

The function of the electrical contact element has been explained above (cf., e.g., the third contact element); it may be integrated in the member or attached thereto. The holding or clamping element may be provided for mechanically interconnecting an element for electrically separating electrical contacts (cf. separating element above) such as a polymer foil to the member, wherein the element may be, e.g., attached to the holding or clamping element, the holding or clamping element being attached to the member or be clamped between the member and the holding or clamping element. The fixing element may be provided for attaching a holding or clamping element to the member and thus may be or at least comprise, e.g., a protrusion or a snap fit portion, so as to firmly seat a holding or clamping element on the member.

The protruding portion may, in particular, hold the electrical contact element.

The electrical contact element may be integrated in the member or fixed to the member.

The attachment to the member may, in particular, be the fixing element.

The attachment to the member may, in particular, hold or accommodate the electrical contact element.

The attachment to the member may, in particular, be the holding or clamping element.

The fixing element may, in particular, be the holding or clamping element.

The device or element may, in particular, be the electrically insulating strip.

In one embodiment, the needle arrangement includes a handle fixed at the needle, wherein the member is the handle.

In another embodiment, the needle arrangement includes a holder or shaft part holding the needle, wherein, the member is the holder or shaft part.

In another embodiment, the needle arrangement includes a needle protection for avoiding access to the needle during and after the removal of the needle from the catheter or cannula.

The invention also includes an electronics unit arrangement for a catheter or cannula arrangement including a catheter or cannula and a sensing element. The invention also includes electronics unit arrangements which includes an electronics unit as described in the present patent application. In particular, the electronics unit arrangement comprises
an electronics unit for processing signals produced by the sensing element and/or for storing data related to signals produced by the sensing element;
an electrical connector for providing an electrical contact to the sensing element;
a housing housing at least a portion of the electronics unit;
one or more fixing structures for attaching the housing to the catheter or cannula.

Such an electronics unit arrangement relates to the above-described special aspect.

The one or more fixing structures may be included in the housing and/or may form a single integrally formed part with at least a portion of the housing. But they may also be distinct parts. The fixing structures can also be considered as fixing elements.

The electrical connector may include, e.g., a (male or a female) plug or socket and/or an electric cable.

The electronics unit usually includes a printed circuit board assembly.

In one embodiment, the electronics unit arrangement includes at least one of
a storage unit for storing data related to signals produced by the sensing element;
electronic circuitry for processing signals produced by the sensing element and/or for obtaining data related to signals produced by the sensing element;
a display for displaying the or other data related to signals produced by the sensing element;
a user control;
an interface, in particular a wire-bound interface, for outputting the or other data related to signals produced by the sensing element, in particular wherein the interface is a digital interface;
a radio frequency transmitter or transceiver for wirelessly transmitting the or other data related to signals produced by the sensing element.

In one embodiment which may be combined with the before-mentioned embodiment, the electronics unit arrangement includes an opening and a contacting element or a separating element extending through the opening operationally connected to at least one electric contact element of the electronics unit, in particular to at least two electric contact elements of the electronics unit.

A contacting element, if present, may in particular be provided for opening an electric contact of the electronics unit in reaction to a movement of the contacting element, in particular in reaction to pulling the contacting element out of the opening.

The separating element, if present, may in particular be provided for closing an electric contact of the electronics unit (which typically is provided by two electrical contact elements) in reaction to a movement of the separating element, in particular in reaction to pulling the separating element out of the opening.

The electronics unit arrangement may include a security unit as described elsewhere in the present patent application.

The contacting element and the separating element may be structured and arranged and used, e.g., like described above.

The invention includes electronics units arrangements with features of corresponding catheter or cannula arrangements according to the invention, and, vice versa, also catheter or cannula arrangements with features of corresponding electronics unit arrangements according to the invention.

The advantages of the electronics unit arrangements basically correspond to the advantages of corresponding catheter or cannula arrangements, and, vice versa, the advantages of the catheter or cannula arrangements basically correspond to the advantages of corresponding electronics unit arrangements.

The invention also includes a method for monitoring a length of stay of a catheter or cannula in a body, wherein the method includes the steps of
a1) providing a catheter or cannula arrangement including a catheter or cannula;
a2) providing a security unit, the security unit including a signalling unit; and including at least one of the steps of
d1) emitting, by means of the signalling unit, a first signal indicative of an attainment of an end of a preset time span;
d2) indicating, by means of the signalling unit, a time corresponding to the length of stay or corresponding to a time remaining up to a preset time span.

In particular, the security unit may be included in the catheter or cannula arrangement.

In one method embodiment, the security unit includes a timer unit, the method furthermore including the step of
c3) measuring, by means of the timer unit, a time duration; and, in particular, also the step of
c4) by means of the timer unit: producing and transmitting to the signalling unit a signal referred to as third signal as soon as the measured time duration is as large as or exceeds the preset time span wherein step d1) is carried out in reaction to receiving the third signal in the signalling unit.

More particularly, in step c3), the time to be indicated according to step d2) may be measured, and/or the time until reaching the preset time span mentioned in step d1) may be measured.

In one method embodiment which may be combined with the before-mentioned embodiment, the security unit includes an activation detection unit, the method including the steps of
b1) detecting, by means of the activation detection unit, a first event;
b2) in reaction to the detecting mentioned in step b1), producing, by means of the activation detection unit, a second signal indicative of the detection.

In one embodiment referring to the two last-mentioned embodiments, the method includes the steps of
c1) receiving, in the timer unit, the second signal;
c2) starting, in reaction to the receiving mentioned in step c1), the time duration measurement mentioned in step c3).

In one method embodiment referring to one or both of the two last-mentioned embodiments, the first event is an at least partial extraction of a needle of the catheter or cannula arrangement from the catheter or cannula.

Of course, the first event can also be a movement or removal of an item (access inhibitor) attached to an access end of a valve device of the catheter or cannula arrangement.

In one method embodiment referring to one or both of the two last-mentioned embodiments, at least one of the following applies
the first signal is an optical signal, in particular an optical signal generated by the security unit; and
the timer unit includes an electronic time piece, in particular wherein the time piece is included in the security unit.

The invention includes methods with features of corresponding security units or of corresponding catheter or cannula arrangements according to the invention, and, vice versa, also catheter or cannula arrangements or security units with features of corresponding methods according to the invention.

The advantages of the methods basically correspond to the advantages of corresponding security units or of corresponding catheter or cannula arrangements, and, vice versa, the advantages of the security units or catheter or cannula arrangements basically correspond to the advantages of corresponding methods.

The invention also includes a method for monitoring a physical quantity sensed by a sensing element of a catheter or cannula arrangement, the method including providing an electronics unit for processing signals produced by the sensing element and/or for storing data related to signals produced by the sensing element, wherein at least one of the following applies:
at least a portion of the electronics unit is present in a housing attached to, in particular fixedly attached to, the catheter or cannula;
at least a portion of the electronics unit, in particular the electronics unit substantially in full, is arranged in a defined position relative to the catheter or cannula, in particular in a fixed position relative to the catheter or cannula.

The above features are usually meant to apply during the monitoring.

Usually, it is provided that the portion of the electronics unit present in the housing and the portion of the electronics unit arranged in the defined position, respectively, is structured and arranged for accomplishing the processing signals produced by the sensing element and/or the storing data related to signals produced by the sensing element.

The physical quantity usually is a physical quantity related to the body to which the catheter or cannula is applied, in particular a physical quantitysensed inside the body. It can be, e.g., temperature.

Further possible features of the method are readily inferred from the electronics units and electronics units arrangements described in the present patent application.

The invention also includes a method for manufacturing a catheter or cannula arrangement, wherein the method includes the step of
A) providing in the catheter or cannula arrangement a security unit for monitoring a length of stay of the catheter or cannula in a body.

In one embodiment, the step A) includes the step of
A1) providing, as a portion of the security unit,
   a printed circuit board assembly including a first and a second electrical contact element;
   a separating element having a first end and distant therefrom a second end, the first end being located between the first and second electrical contact elements, electrically separating these from each other, the second end being provided with a fixing element for fixing the second end to another portion of the catheter or cannula arrangement.

Usually, the fixing element is a part not integrally formed with the separating element, but a part manufactured separately from the separating element which is attached to the second end of the separating element.

In one embodiment referring to the last-mentioned embodiment, the catheter or cannula arrangement includes a needle arrangement separable from the catheter or cannula, and the method includes the step of attaching the second end to a constituent part of the needle arrangement by fixing the fixing element to the constituent part.

The constituent part may be, e.g., a handle of the needle arrangement. The fixing of the fixing element to the constituent part may, e.g., be a sliding of the fixing element onto the constituent part or a pushing of the fixing element into the constituent part. The fixing element may have a snap fit connection with the constituent part.

The invention includes manufacturing methods with features of corresponding security units or of corresponding catheter or cannula arrangements or of corresponding methods for monitoring according to the invention, and, vice versa, also catheter or cannula arrangements or security units or methods for monitoring with features of corresponding manufacturing methods according to the invention.

The advantages of the manufacturing methods basically correspond to the advantages of corresponding security units or of corresponding catheter or cannula arrangements or of corresponding methods of monitoring, and, vice versa, the advantages of the security units or catheter or cannula arrangements or methods of monitoring basically correspond to the advantages of corresponding manufacturing methods.

The invention also includes devices, wherein a first type of device is particularly suitable for realizing integrated solutions, and a second type of devices is particularly suitable for realizing retrofit solutions.

Thus, the invention also includes a device (of the first type) including a cap for sealing an injection port of a catheter or cannula arrangement, the device including a member forming the cap, the member including:
a first part for at least partially housing and/or at least contributing to locating a printed circuit board;
a second part attached to or attachable to the first part, for plugging the injection port and/or for at least partially housing and/or at least contributing to locating a printed circuit board;
a third part attached to or attachable to the second part, for fastening the member to another portion of a catheter or cannula arrangement, in particular to the injection port.

Therein, the member may in particular be integrally formed, in particular by injection molding. And the device may include a printed circuit board arranged, at least in part, in the member.

And, the invention also includes a device (of the second type) including an attachment to a cap for sealing an injection port of a catheter or cannula arrangement, the device including a member forming the attachment to a cap, the member including:
a first part for at least partially housing and/or at least contributing to locating a portion of a printed circuit board; and
a second part attached to or attachable to the first part, for attaching the attachment to the cap and/or for at least partially housing and/or at least contributing to locating a printed circuit board.

Also this member may in particular be integrally formed, e.g., by injection molding; and the device may include a printed circuit board arranged, at least in part, in the member.

And, the invention also includes a device including an extension connectable, at its first end, to a connection end of a catheter or cannula arrangement, the extension providing an inner channel for flow of a fluid through the catheter or cannula. And the extension provides an inner volume separate from the inner channel for at least partially housing and/or at least contributing to locating a printed circuit board. And, the extension may, at its second end, provide a connector for connecting a device by which the fluid is supplied or removed, such as a syringe or a tube.

The extension itself may be the device.

The device or the extension may be provided by one or more, typically one or two, integrally formed parts such as injection molded parts.

The device may have further features as disclosed in the present patent application, in particular as described for the extension.

And, the invention also includes a device including an attachment to a valve device of a catheter or cannula arrangement, the device including a first part for fastening the device to the valve device and, interconnected to the first part, a second part providing a housing, the first part having at a first end a snap fit portion and providing between the first end and a second end an inner volume extending generally along an axis for accommodating at least a portion of the valve device.

Therein, first part and second part may be distinct parts or may, in particular, form a single integrally formed part.

The first part may have a generally tubular or cylindrical shape.

The first end of the first part can be or provide a snap fit portion for interacting with an edge of the valve device.

At the first end of the first part, two or more legs may be provided, in particular legs having inwardly pointing protrusions at the first end.

At the second end of the first part (which typically is arranged opposed to the first end) the attachment may have an opening allowing another portion of the valve device to protrude.

And, at the second end of the first part, there may be an inner surface facing towards the first end functioning as a mechanical stop for portion of the valve device.

It can in particular be provided that at least a portion of the valve device is clamped between the inner surface and the second end, more particularly before-mentioned inwardly pointing protrusions at the first end.

The second part may present at an outside perimeter of the first part.

The attachment to a valve device itself may be the device.

The device may have further features as disclosed in the present patent application, in particular as described for the attachment to a valve device.

And, the invention also includes uses, more particularly uses of an assembly. One use is a use of an assembly including:
a printed circuit board assembly including a first and a second electrical contact element;
a separating element having a first end and a second end, the first end being located between the first and second electrical contact elements, electrically separating these from each other, the second end being provided with a fixing element for fixing the second end to another member or element;
   as a security unit for monitoring a length of stay of a catheter or cannula in a body or as a portion of such a security unit.

Therein, the first and a second electrical contact elements may in particular form a spring-loaded contact.

Another use is a use of an assembly for the purpose of monitoring a length of stay of a catheter or cannula in a body, wherein the assembly includes:
a printed circuit board assembly including a first and a second electrical contact element and embodying a timer unit;
a separating element having a first end and a second end, the first end being located between the first and second electrical contact elements, electrically separating these from each other, the second end being provided with a fixing element for fixing the second end to another member or element;
   wherein engaging the first and second electrical contact elements results in the timer unit starting a time measurement.

In addition, it may be provided that the catheter or cannula arrangement has RFID capability. In other words, in this case, the catheter or cannula arrangement has a data storage containing identification data which are data for identifying the catheter or cannula arrangement or at least a component thereof. And, in addition, the catheter or cannula includes a radio frequency transmitter of transceiver for transmitting the identification data. It is possible that the energy required for such a transmission of data is provided by the catheter or cannula arrangement itself, but it may also be provided that the energy required for such a transmission of data is provided by an external device such as a reader (RFID reader). A result of the latter can be that an energy storage, e.g., a battery, of the catheter or cannula can last longer.

The RFID capability can simplify the handling of the catheter or cannula and in particular handling data produced by the catheter or cannula arrangement such as data related to the dwell time and/or data related to a sensed physical quantity (in case a sensing element is included in the catheter or cannula arrangement). For example, the data produced by the catheter or cannula arrangement can be tagged with the identification data.

For example, the catheter or cannula arrangement is structured and arranged such that in reaction to receiving a request signal (which may be constituted by wirelessly receiving data indicative of a request, or by operating a user control of the catheter or cannula arrangement), the identification data are transmitted. For example, a (mobile) reader, typically operated by a health care professional, can emit a request signal and receive the identification data, e.g., together with further data produced by the catheter or cannula.

The transmitter or transceiver may be identical with (or different from) a transmitter or transceiver used for wirelessly transmitting other data such as the before-mentioned data related to a sensed physical quantity and/or data related to the dwell time.

The transmitter or transceiver and/or the before-mentioned data storage may be included in an security unit and/or in an electronics unit.

Further embodiments and advantages emerge from the dependent claims and the figures.

### BRIEF DESCRIPTION OF THE DRAWINGS

Below, the invention is described in more detail by means of examples and the included drawings. The figures show:

FIG. 1 a perspective view of a catheter or cannula arrangement;

FIG. 2 a perspective exploded view of the catheter or cannula arrangement of FIG. 1;

FIG. 3 a perspective view of a portion of a security unit of the arrangement of FIG. 1;

FIG. 4 a perspective view of a cap including a portion of a security unit;

FIG. 5 a bottom view of a cap including a portion of the security unit of FIG. 4;

FIG. 6 a perspective view of a detail of a catheter or cannula arrangement of FIG. 1;

FIG. 7 a perspective view of a detail of the catheter or cannula arrangement of FIG. 1;

FIG. 8 a perspective view of a detail of a needle arrangement;

FIG. 9 a perspective view of an electrical contact element of FIG. 8;

FIG. 10 a cross-sectional view of a detail of the catheter or cannula arrangement of FIG. 1;

FIG. 11 a schematic block-diagrammatical illustration of a catheter or cannula arrangement including a security unit.

FIG. 12 a perspective view of a further catheter or cannula arrangement;

FIG. 13 a perspective view of a detail of a the arrangement of FIG. 12;

FIG. 14 a perspective view of an attachment to a cap of the arrangement of FIG. 12;

FIG. 15 a further perspective view of an attachment to a cap of the arrangement of FIG. 12;

FIG. 16 a perspective view of a security unit of a catheter or cannula arrangement, more particularly of an attachment to a handle of a needle arrangement;

FIG. 17 a perspective view of a detail of a security unit of a catheter or cannula arrangement;

FIG. 18 a further perspective view of a detail of the security unit of FIG. 17;

FIG. 19 a perspective view of a further detail of the security unit of FIG. 17;

FIG. 20 a perspective view of another catheter or cannula arrangement with partially removed needle;

FIG. 21 a perspective view of the catheter or cannula arrangement of FIG. 20 with inserted needle;

FIG. 22 a rear view of the catheter or cannula arrangement of FIG. 20 with inserted needle;

FIG. 23 a cross-sectional view of the catheter or cannula arrangement of FIG. 20 with inserted needle;

FIG. 24 a rear view of the catheter or cannula arrangement of FIG. 20 with partially removed needle;

FIG. 25 a cross-sectional view of the catheter or cannula arrangement of FIG. 20 with partially removed needle;

FIG. 26 a perspective view of the cap of the catheter or cannula arrangement of FIG. 20 including a portion of a security unit;

FIG. 27 a rear view of the cap of the catheter or cannula arrangement of FIG. 20 including a portion of a security unit;

FIG. 28 a cross-sectional view of the cap of the catheter or cannula arrangement of FIG. 20 including a portion of a security unit;

FIG. 29 an enlarged detail of the cross-sectional view of FIG. 23;

FIG. 30 an enlarged detail of the cross-sectional view of FIG. 25;

FIG. 31 a perspective view of the member forming the cap of the catheter or cannula arrangement of FIG. 20;

FIG. 32 a perspective view of the member forming the cap of the catheter or cannula arrangement of FIG. 20;

FIG. 33 a rear view of the member forming the cap of the catheter or cannula arrangement of FIG. 20;

FIG. 34 an axial cross-sectional view of the member forming the cap of the catheter or cannula arrangement of FIG. 20;

FIG. 35 a side view of the member forming the cap of the catheter or cannula arrangement of FIG. 20;

FIG. 36 a radial cross-sectional view of the member forming the cap of the catheter or cannula arrangement of FIG. 20;

FIG. 37 a perspective view of the member forming the cap of the catheter or cannula arrangement of FIG. 20 in unfolded state;

FIG. 38 a cross-sectional view of the member forming the cap of the catheter or cannula arrangement of FIG. 20 in unfolded state;

FIG. 39 a cross-sectional view of a further catheter or cannula arrangement with inserted needle;

FIG. 40 a cross-sectional view of the catheter or cannula arrangement of FIG. 39 with partially removed needle;

FIG. 41 a perspective view of the cap and attachment to the cap of the catheter or cannula arrangement of FIG. 39, the attachment including a portion of a security unit;

FIG. 42 a radial side view of the cap and attachment to the cap of FIG. 41;

FIG. 43 an enlarged detail of the cross-sectional view of FIG. 39;

FIG. 44 an enlarged detail of the cross-sectional view of FIG. 40;

FIG. 45 a rear view of the cap and attachment to the cap of the catheter or cannula arrangement of FIG. 39;

FIG. 46 a cross-sectional view of the cap and attachment to the cap of the catheter or cannula arrangement of FIG. 39 including a portion of a security unit;

FIG. 47 a perspective view of the attachment to the cap of the catheter or cannula arrangement of FIG. 39;

FIG. 48 a radial side view of the attachment to the cap of the catheter or cannula arrangement of FIG. 39;

FIG. 49 an axial end view of the member forming the attachment to the cap of the catheter or cannula arrangement of FIG. 39 in unfolded state;

FIG. 50 a cross-sectional view of the member forming the attachment to the cap of the catheter or cannula arrangement of FIG. 39 in unfolded state;

FIG. 51 a perspective view of the member forming the attachment to the cap of the catheter or cannula arrangement of FIG. 39 in unfolded state;

FIG. 52 a perspective view of the electrical contact element of the security unit of the catheter or cannula arrangement of FIG. 20 or of FIG. 39;

FIG. 53 a cross-sectional view of the attachment to the handle of the catheter or cannula arrangement of FIG. 20 or of FIG. 39;

FIG. 54 a perspective view of the attachment to the handle of the catheter or cannula arrangement of FIG. 20 or of FIG. 39;

FIG. 55 a perspective view of another catheter or cannula arrangement with inserted needle;

FIG. 56 a perspective view of the catheter or cannula arrangement of FIG. 55 with partially removed needle;

FIG. 57 a perspective view of constituents of the catheter or cannula arrangement of FIG. 55;

FIG. 58 an illustration of a portion of another catheter or cannula arrangement, with time display;

FIG. 59 a top view of a detail of a catheter or cannula arrangement with pull-out tab, with inserted needle;

FIG. 60 a side view of the detail of the catheter or cannula arrangement of FIG. 59;

FIG. 61 a perspective view of the detail of the catheter or cannula arrangement of FIG. 59, the attachment to the cap not shown;

FIG. 62 a perspective view of an enlarged detail of the catheter or cannula arrangement of FIG. 59, the attachment to the cap not shown;

FIG. 63 a perspective approximate top view of a detail of the catheter or cannula arrangement of FIG. 59, the attachment to the cap not drawn;

FIG. 64 a schematic illustration of a portion of a security unit with pull-out tab in an initial position, in a side view;

FIG. 65 a schematic illustration of the portion of the security unit of FIG. 64, with pulled-out pull-out tab, in a side view;

FIG. 66 a perspective view of a catheter or cannula arrangement including an extension;

FIG. 67 a perspective view of a detail of the catheter or cannula arrangement of FIG. 66, further including a closure or cover;

FIG. 68 a perspective view of a detail of the catheter or cannula arrangement of FIG. 66, further including a needle arrangement;

FIG. 69 a perspective view of the extension of FIGS. 66 to 68;

FIG. 70 a perspective view of the extension of FIGS. 66 to 69, with a removed housing part;

FIG. 71 a perspective view of the extension of FIG. 70 with inserted printed circuit board assembly;

FIG. 72 a perspective view of a detail of a catheter or cannula arrangement including a valve device to which a security unit is connected;

FIG. 73 another perspective view of a detail of the catheter or cannula arrangement of FIG. 72;

FIG. 74 a perspective view of a detail of the catheter or cannula arrangement of FIGS. 72 and 73 with all parts of the security unit removed;

FIG. 75 a perspective view of a detail of the catheter or cannula arrangement of FIG. 74 with attachment to valve device attached;

FIG. 76 a perspective view of a detail of the catheter or cannula arrangement of FIG. 74 with attachment to valve device and closure attached;

FIG. 77 a perspective view of a detail of a catheter or cannula arrangement known in the art, including a sensing element;

FIG. 78 a perspective view of a catheter or cannula arrangement including an electronics unit;

FIG. 79 a perspective view approximately onto the front of a detail of a catheter or cannula arrangement including an electronics unit;

FIG. 80 a perspective view approximately onto the back of a detail of the catheter or cannula arrangement of FIG. 79.

The described embodiments are meant as examples and shall not limit the invention.

### DETAILED DESCRIPTION OF THE INVENTION

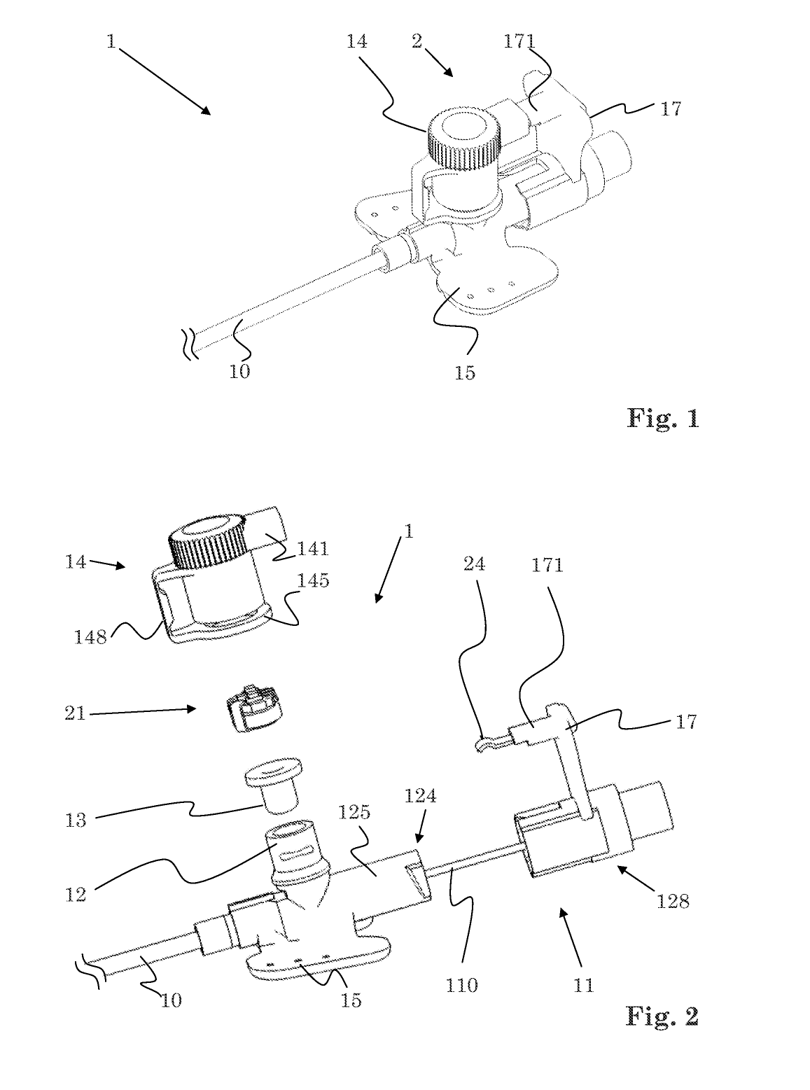

FIG. 1 is a perspective view of a catheter or cannula arrangement 1, FIG. 2 is a perspective exploded view of the same. The arrangement 1 includes a catheter or cannula 10, a needle arrangement 11, an injection port 12, wings 15, a cap 14 attached to injection port 12 by a cap fastener 145 connected to cap 14 by a flexible interconnecting part 148, and a seal or plug 13 for sealing injection port 12. Needle arrangement 11 includes a needle 110 and a handle 17 including a protruding portion 171. The wings 15 are provided on a shaft part 125 which may also be a holder for holding the catheter or cannula 10, and shaft part 125 has an end 124 which will be referred to as connection end 124 to which further devices such as a syringe or a tube can be connected (after removal of the needle 11).

Arrangement 1 also includes a security unit 2 a portion of which is provided in needle arrangement 11, whereas another portion of security unit 2 (typically the rest of security unit 2) is provided in cap 14, in particular a printed circuit board assembly 21 (PCBA 21).

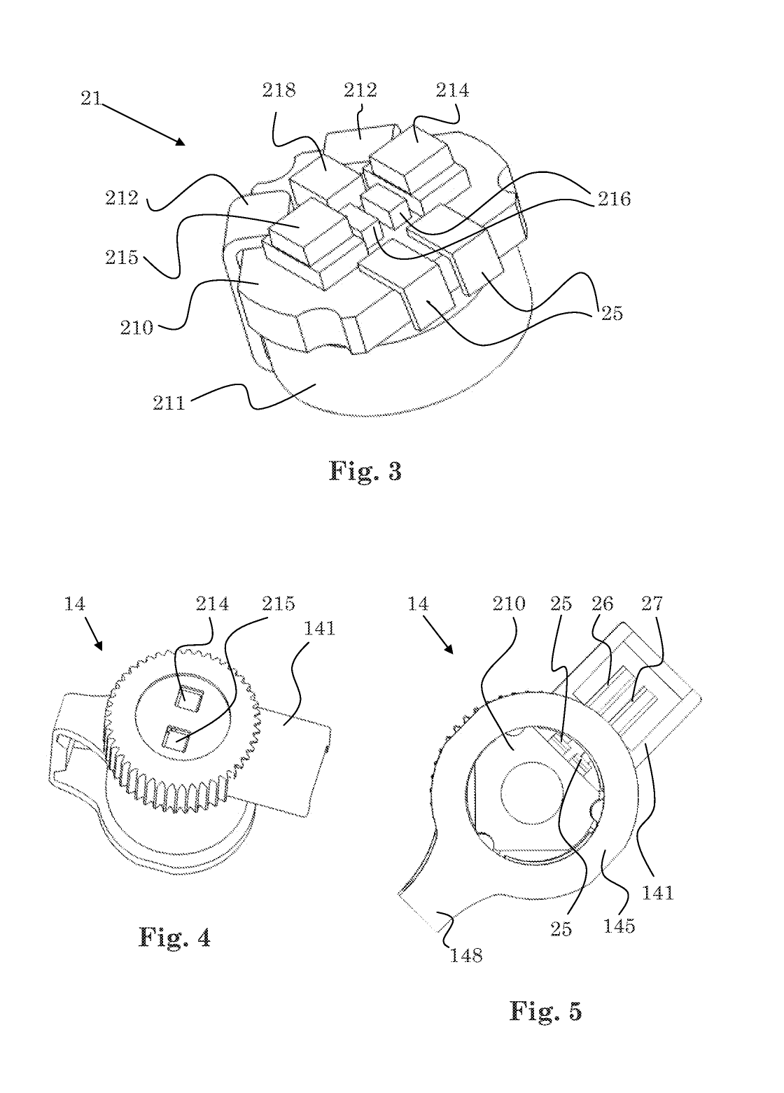

FIG. 3 is a perspective view of a portion of security unit 2. It basically illustrates printed circuit board assembly 21 (PCBA 21). PCBA 21 includes a printed circuit board 210 (PCB 210), a battery 211, battery contacts 212, capacitors 216, an integrated circuit 218, two light emitters 214, 215, e.g., LEDs (light emitting diodes), and two electrical contact elements such as contact leads 25. All these components of PCBA 21 are operationally interconnected.

FIG. 4 is a perspective view of cap 14 including a portion of a security unit 2, e.g., of the one of the catheter or cannula arrangement of FIGS. 1 and 2. A protruding portion 141 of cap 14 is visible. LEDs 214, 215 are visible through openings or transparent portions in cap 14.

FIG. 5 is a bottom view of cap 14 including a portion of a security unit 2. Further electrical contact elements of security unit 2 are visible, more particularly contact pins 26, 27 which are each in galvanic contact with one of contact leads 25.

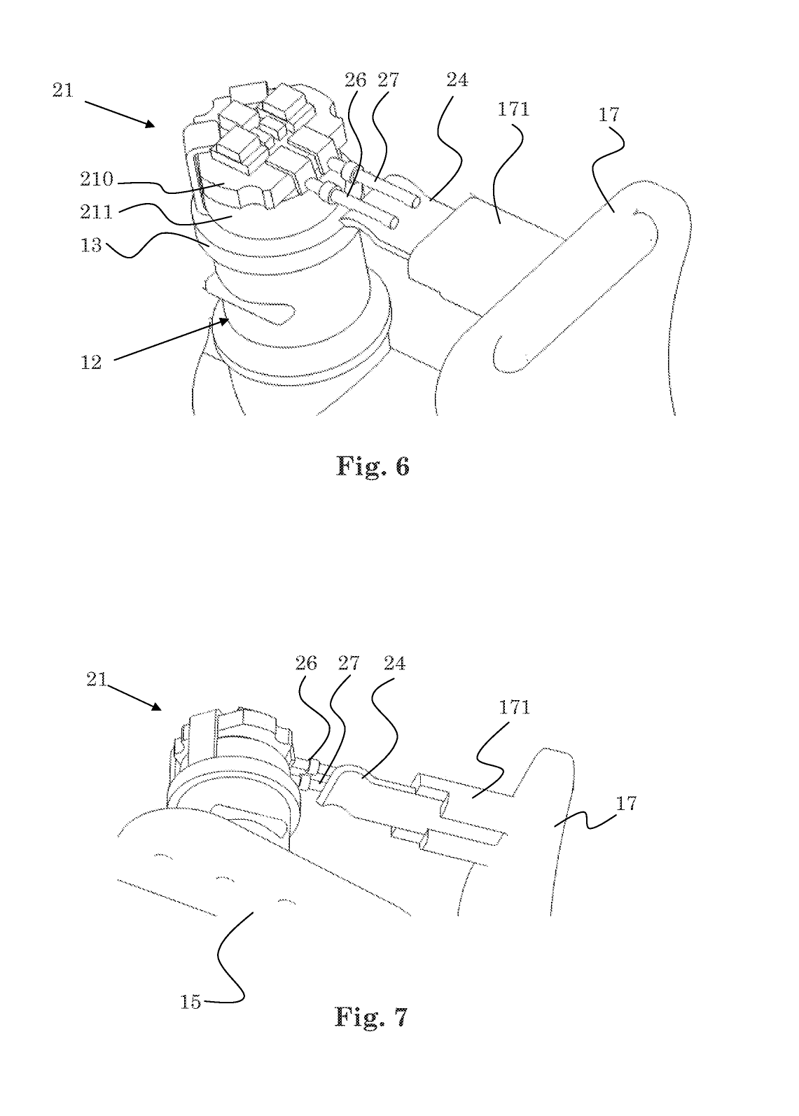

FIGS. 6 and 7 are top and bottom perspective views of a detail of the catheter or cannula arrangement 1. Plug 13 seals or separates PCBA 21 from port 12 preventing PCBA 21 from being exposed to fluids flowing through catheter or cannula 10, such as blood and pharmaceuticals. In the situation illustrated in FIG. 6, needle 110 (cf. FIG. 2) is fully pushed into catheter or cannula 10, usually such that a tip of needle 110 protrudes from catheter or cannula 10. In this situation, contact pins 26 and 27 are in galvanic contact with sheet metal tongue 24. Thus, sheet metal tongue 24 closes an electrical contact between contact pins 26 and 27 and thus also between the two contact leads 25. Note that FIGS. 6 and 7 are merely schematical illustrations and that in reality, where contact pins 26, 27 are in touch with sheet metal 24, sheet metal tongue 24 is not locally deformed in the illustrated way, but sheet metal tongue 24 will rather bend slightly.

FIG. 7 is a perspective view of a detail of a catheter or cannula arrangement 1.

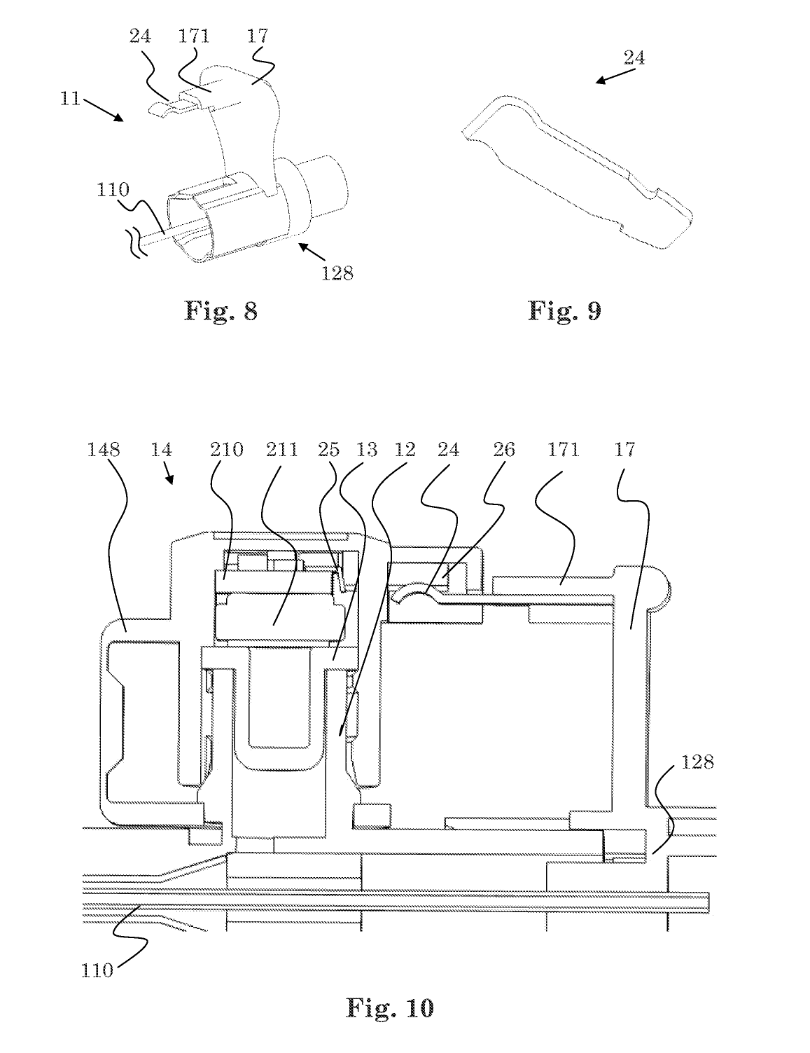

FIG. 8 is a perspective view of a detail of a needle arrangement 11, e.g., of the one of the catheter or cannula arrangement of FIGS. 1 and 2. Handle 17 facilitates moving needle 110 with respect to catheter or cannula 10.

FIG. 9 is a perspective view of an electrical contact element, such as the one of FIG. 8, more particularly of sheet metal tongue 24. Indents at the sides of sheet metal can facilitate fixing sheet metal tongue 24 to handle 17, more particularly to protrusion 171 thereof. In protrusion 171, corresponding protrusions cooperating with the indents can be provided.

FIG. 10 is a cross-sectional view of a detail of a catheter or cannula arrangement 1 including security unit 2.

In the following, it is described how security unit 2 can be used for monitoring a length of stay of catheter or cannula 10 in a body, thus facilitating to prevent a too long stay of catheter or cannula 10 in the body, which may be a human or an animal's body.

Before use, when delivered, catheter or cannula arrangement 1 is, e.g., configured as shown in FIG. 1. Needle 110 (cf. FIG. 2) is, at least in part (and usually as far as possible) located inside catheter or cannula 10. And sheet metal tongue 24 bridges the two contact pins 26 and 27. The latter fact can be sensed by integrated circuit 218 (e.g., an ASIC, i.e. an application specific integrated circuit), e.g., by sensing an electrical resistance between the two contact leads 25 and determining whether it is smaller than specific preset threshold resistance or not.

After catheter or cannula 10 has been inserted into a body, e.g., more particularly into a vein pierced by the needle 110, needle arrangement 11 is removed from the rest of arrangement 1, usually by pulling handle 17 parallel to the axis defined by catheter or cannula 10. This way, the formerly closed electrical contact between contact leads 25 is broken by sheet metal tongue 24 sliding along and then off contact pins 26, 27. The open (broken) contact can be sensed by integrated circuit 218 (cf. FIG. 3).

Since the pulling-out of the needle 110 is usually carried out shortly after inserting the catheter or cannula 10 in a body, the sensing of the open contact can be used as an event initiating a time measurement allowing to monitor a length of stay of the catheter or cannula 1 in the body. A timer and control logic for accomplishing this may be implemented in integrated circuit 218. Depending on the length of time for which the contact is open or closed, LEDs 214, 215 can emit different signals, in particular different light pulses or different patterns of light pulses. It may also be provided that emitted signals also depend on a time interval during which the contact is open before it is possibly closed again and/or during which the contact is closed before it is opened again. This may be helpful, e.g., for testing the security unit.

In the following description of a method, it may be assumed that LED 214 emits green light and LED 215 emits red light.

Initially, after assembly of PCBA 21, the contact is open, and security unit 2 is in an idle mode in which no signals are emitted. This saves energy.

A test mode may be entered, if the contact is closed for less than 10 seconds. In the test mode, a light pulse pattern may be emitted, in which both LEDs 214, 215 are activated.

In reaction to a subsequent detection that the contact is closed for more than 10 seconds, a triggered mode is entered in which a light pulse pattern using LED 214 only may be emitted. Then, the catheter or cannula arrangement 1 can be packed and prepared for use.

When thereafter, the removal of the needle 110 takes place, it is sensed that the contact is open, and a signal (more particularly: a pulse pattern) using LED 214 only is emitted, e.g., by emitting a rapid sequence of light pulses (e.g., pulses following each other in a distance of at least 0.1 s and at most 5 s) followed by a sequence of light pulses following each other in a larger time distance, e.g., of at least 10 s and at most 120 s. That pulse pattern shall indicate that the use of the catheter or cannula arrangement 1 is safe (safe operation mode), and it is continued until a preset time span (e.g., 72 hours) has passed.

Thereafter, a replacement warning mode is entered during which a signal (more particularly: a pulse pattern) is emitted involving both LEDs 214, 215, e.g., LED 214 continuing as before (in the safe operation mode), and LED 215 emitting a light pulse immediately after the end of each pulse of LED 214. This shall indicate that the catheter or cannula has to be replaced soon in order to minimize the probability of an infection or other problems because of catheter or cannula 10 being in the body for a too long time. This mode is continued for another preset time period such as 24 hours.

After that, an end-of-life mode is entered and continued (until exhaustion of battery 211) which shall indicate that the time for removing catheter or cannula 1 from the body has already passed. A signal (more particularly: a pulse pattern) is emitted during this mode using LED 215 only, e.g., emitting pulses in a distance of at least 1 s to at most 20 s.

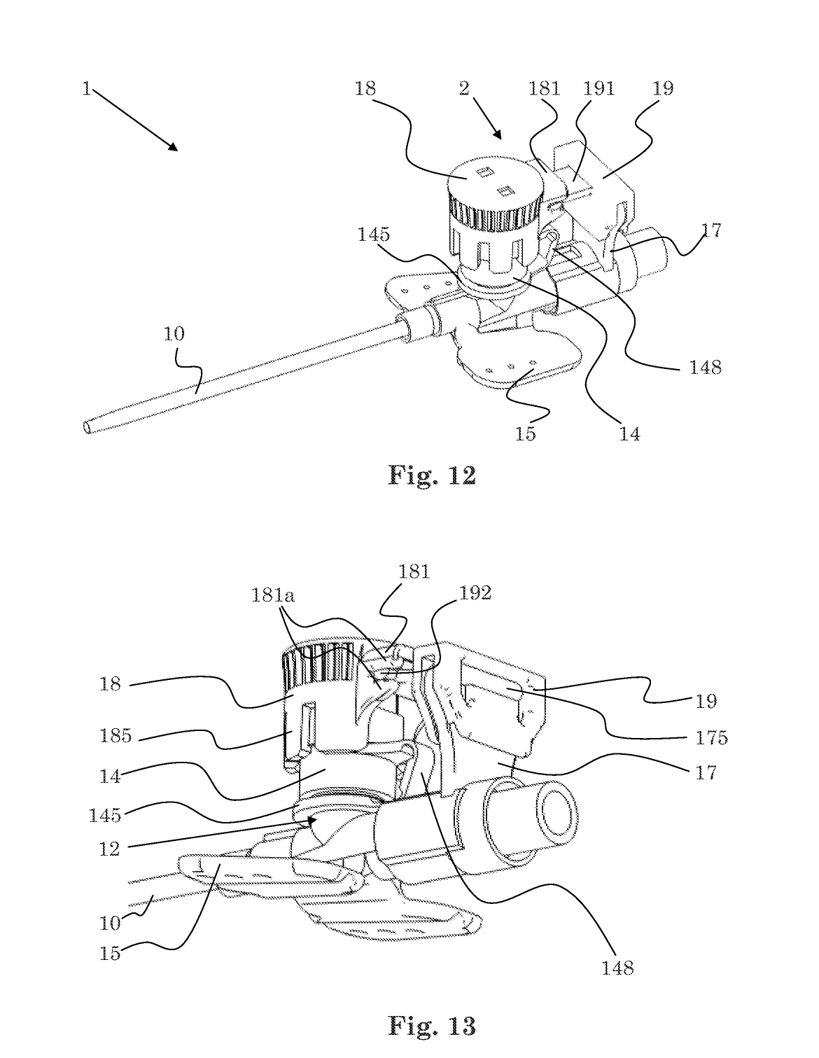

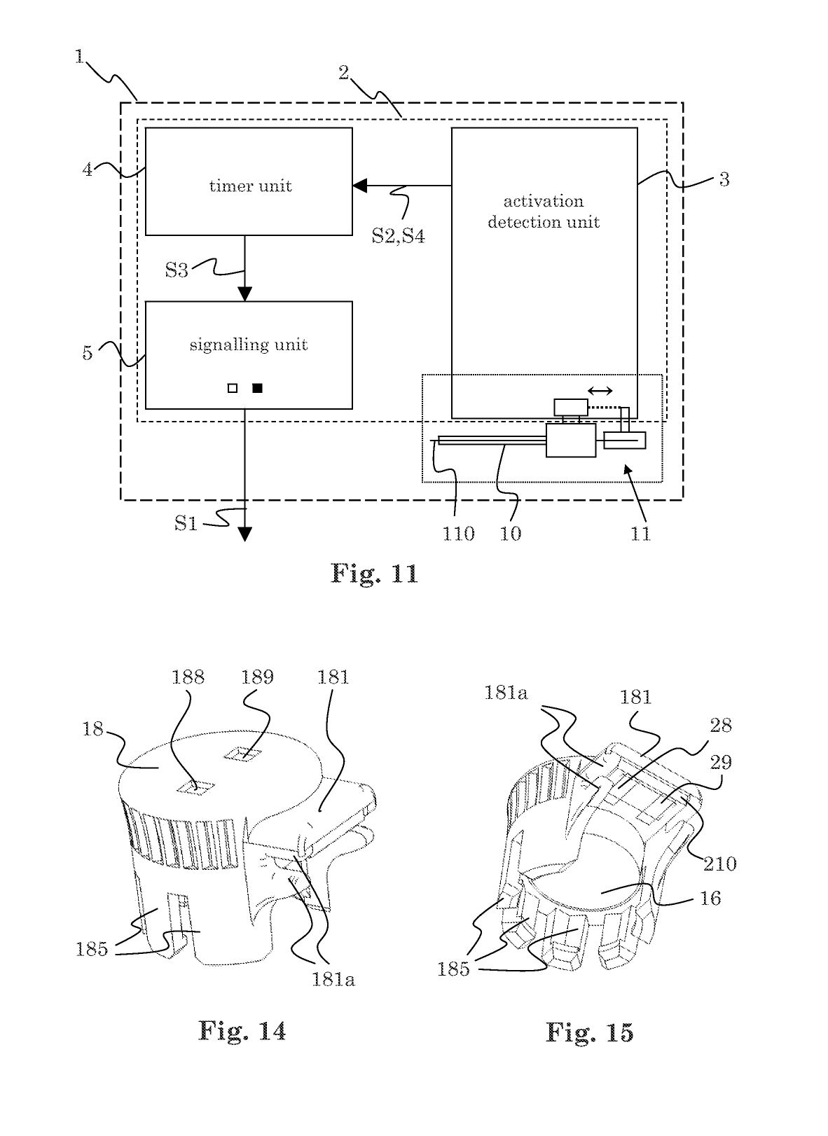

FIG. 11 is a schematic block-diagrammatical illustration of a catheter or cannula arrangement 1 including a security unit 2, emphasizing functional aspects. FIG. 11 in particular illustrates operation of a catheter or cannula arrangement such as of the one of FIGS. 1 and 2. Security unit 2 includes an activation detection unit 3, a timer unit 4 and a signalling unit 5. Timer unit 4 can at the same time also be a control unit, but one could also provide a timer unit 4 and, in addition, a control unit.

Activation detection unit 3 allows detection of an event in reaction to which the above-described safe-operation mode shall be entered. One portion of activation detection unit 3 is included in needle arrangement 11, in particular sheet metal tongue 24 (cf. FIG. 10). Another portion of activation detection unit 3 is included in cap 14 (cf. FIG. 10), namely in particular contact pins 26, 27 and a portion which can be embodied in integrated circuit 218, namely, e.g., a detector for electrical resistances (which merely needs to be able to distinguish open from closed electrical contacts).

A signal S2 is sent by activation detection unit 3 to timer unit 4 when the contact (between contact pins 26 and 27, bridged beforehand by sheet metal 24) is opened. Thus, signal S2 is indicative of a removal of the needle 110 (first event).

A signal S4 is sent by activation detection unit 3 to timer unit 4 when the contact (between contact pins 26 and 27) is closed (after having been open before). Thus, signal S4 is indicative of a re-insertion of the needle into catheter or cannula 10 (second event).

Timer unit 4 may be substantially included in integrated circuit 218 and allows determination of an amount of time that passed after having received signal S2 or S4 from activation detection unit 3. It thus can measure the time that passed after the occurrence of a first or a second event. Timer unit 4 can compare a measured time span with a preset time span and produce and transmit to signalling unit 5 a signal S3 in case the preset time span is reached or exceeded.

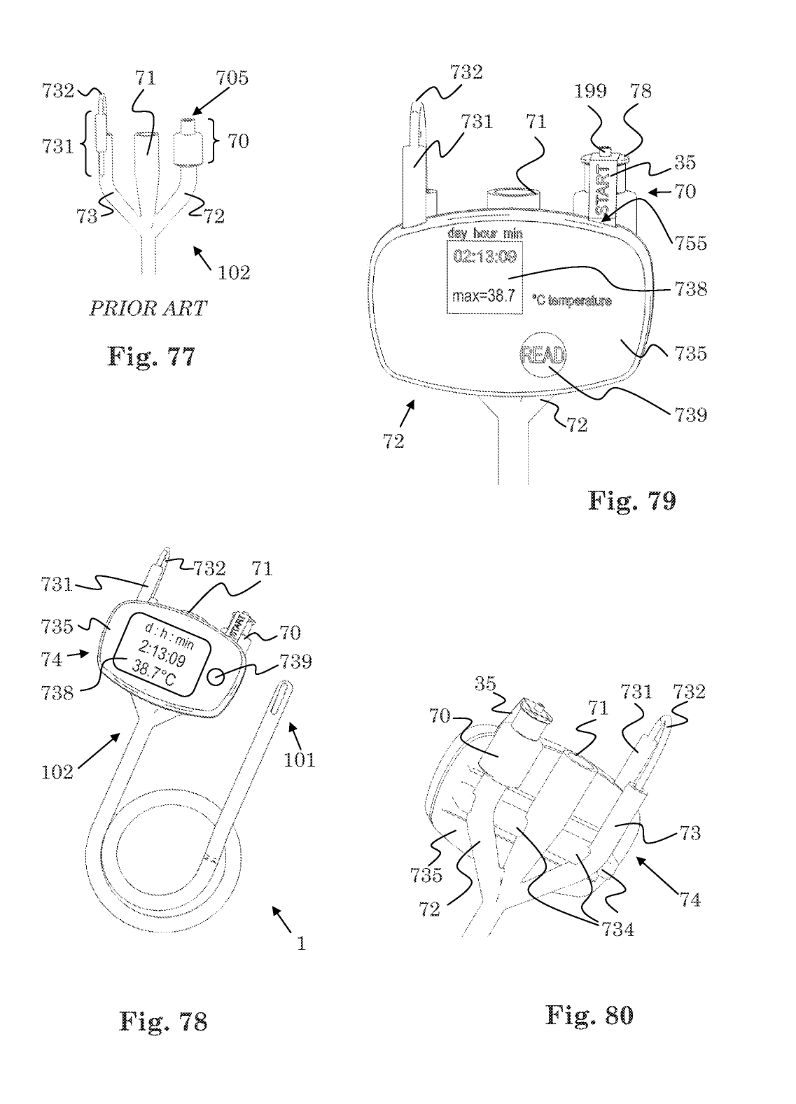

Signalling unit 5 includes LEDs 214, 215 and may in part be embodied in integrated circuit 218, namely for accomplishing that various pulse patterns can be emitted by LEDs 214, 215. Signalling unit 5 allows a person to determine (from the signals emitted by signalling unit 5) whether or not catheter or cannula 10 has to be removed from the body (as estimated from the length of time catheter or cannula 10 already has remained in the body, which again is estimated from the time that passed since removal of needle 110 from catheter or cannula 10). As will become clear, a signalling unit 5 as described and at least portions of PCBA 21 can also be used in conjunction with the special aspect (sensing a physical quantity using the catheter or cannula arrangement), cf. also below FIGS. 77 to 80.

The embodiments illustrated in FIGS. 1 to 10 are not compatible with today's commercially available catheter or cannula arrangements. The illustrated catheter or cannula arrangements 1 of FIGS. 1 to 10 or at least portions thereof, such as protruding portions 141, 171, are specifically designed for embodying one or more aspects of the invention.

Therefore, alternative embodiments are described in relation to FIGS. 12 to 19, which allow a security unit 2 to be retrofitted to currently commercially available catheter or cannula arrangements. Otherwise, features (physical and functional features) of these embodiments are predominantly identical with those discussed above.

In particular, PCBA 21 is not included in cap 14 of injection port 12, but in an attachment 18 to cap 14. Instead of providing a protruding portion 141 of cap 14, a protruding portion 181 of attachment 18 is provided. And instead of providing protruding portion 171 of handle 17, an attachment 19 to handle 17 is provided which again includes a protruding portion 191. Handle 17 includes a protrusion or an enlargement 175 interacting with attachment 19 to handle 17, for fixing attachment 19 to the handle 17, e.g., forming a snap fit.

FIG. 12 is a perspective view of such a catheter or cannula arrangement 1, and FIG. 13 is a perspective view of a detail of the catheter or cannula arrangement of FIG. 12. Jaws 181a formed by protruding portion 181 are visible in FIG. 13. They provide, via protrusions 192, guidance for protruding portion 191 of attachment 19 to handle 17 being inserted into protruding portion 18 and may also fix protruding portion 191 thereto.

FIG. 14 is a perspective view of attachment 18 to a cap 15 of a catheter or cannula arrangement 1. FIG. 15 is another perspective view of an attachment 18 to a cap 14 of a catheter or cannula arrangement 1. Attachment 18 has openings or transparent portions 188, 189 through which the LEDs can emit light and includes a number of legs 185 with inwardly-directed projections at their free ends by means of which attachment 18 can be attached to cap 14 in a simple but reliable way. The projections at the ends of the legs 185 snap over corresponding portions of an edge of cap 14 (not illustrated), so that the projections engage under the cap 14.

In order to prevent a degradation of PCBA 21 (and thus replacing plug 13, cf. FIG. 2) a portion of PCBA 21 is molded, e.g., covered by a resin. A corresponding casting 16 is shown in FIG. 15. PCB 210 includes contact leads 28, 29 as electrical contact elements.

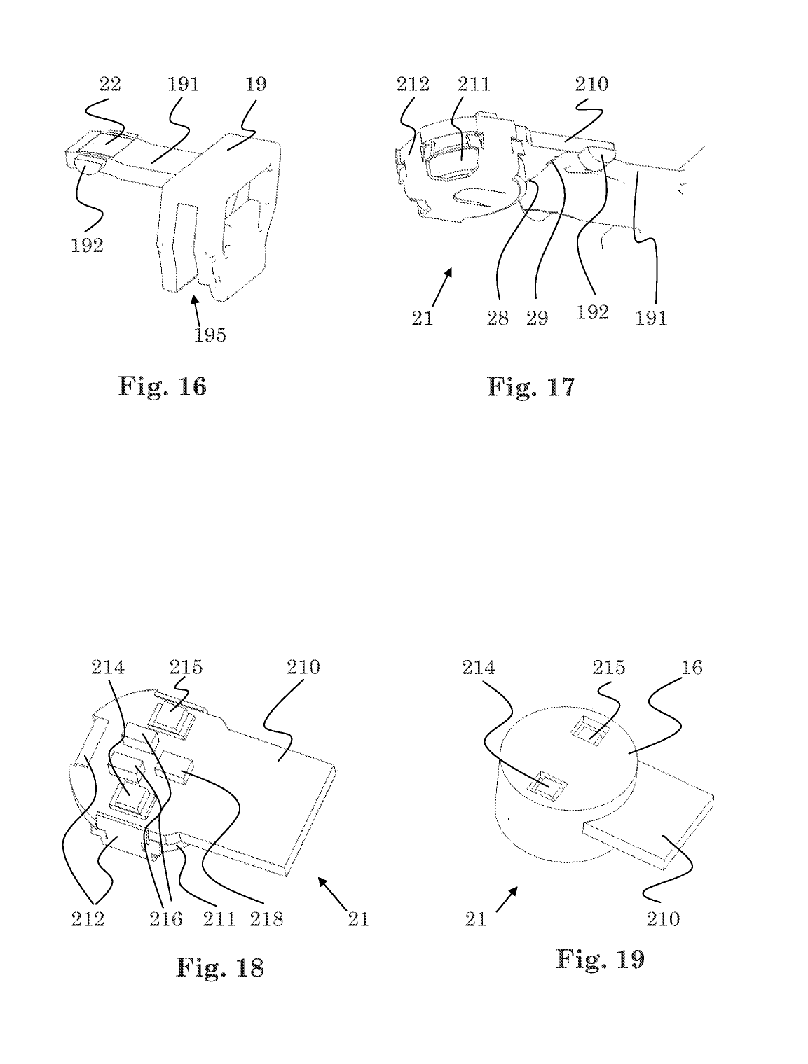

FIG. 16 is a perspective view of a portion of a security unit 2 of a catheter or cannula arrangement 1 such as the one of FIG. 12, more particularly of an attachment 19 to a handle 17 of a needle arrangement 11. Attachment 19 may be fixed to handle 17, e.g., by a snap fit. A groove or channel 195 is provided in attachment 19 for accepting a handle 17. Furthermore, protruding portion 191 can be made predominantly of an electrically insulating material such as a polymer, and for bridging contacts (such as contact leads 28, 29, cf. FIGS. 15 and 17), an electrical contact element 22 such as a coating, in particular a metal coating, can be provided, or a sheet metal can be applied on protruding portion 191. Protruding portion 191 includes two portions 192, e.g., as illustrated, of semicircular cross-section, for interacting with jaws 181a (cf. FIGS. 13-15).

FIG. 17 is a perspective view of a detail of a security unit 2 of a catheter or cannula arrangement 1, e.g., of the one of FIG. 12. Electrical contact elements 28, 29 in form of contact leads 28, 29, e.g., manufactured as a portion of printed circuit board 210 (PCB 210), are visible. Whether or not they are bridged (e.g., by electrical contact element 22) can be sensed.

FIG. 18 is a perspective view of a detail of a security unit 2 of a catheter or cannula arrangement 1. The constituents have been described above.

FIG. 19 is a perspective view of a detail of a security unit 2 of a catheter or cannula arrangement 1, such as the one of FIG. 12. A portion of PCBA 21 is shown in FIG. 19 in a casting 16. An excellent protection from environmental influences for components of PCBA 21 can be achieved this way, in particular for battery 211. An alternative to a casting is a conformal coating. Conformal coatings are a known way of protecting printed circuit board assemblies or parts thereof, e.g., from environmental influences such as humidity.

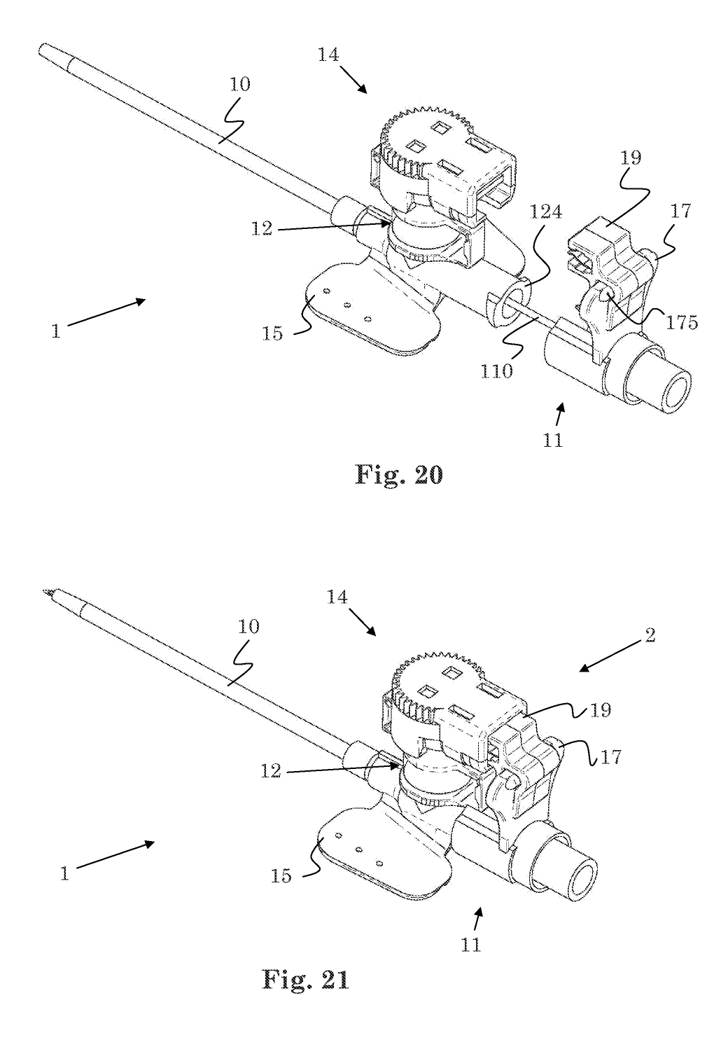

FIGS. 20 to 38 illustrate another catheter or cannula arrangement 1 which in many functional aspects is similar to the before-described ones. It realizes, like the one of FIG. 1, an integrated solution for the cap 14, and, like the one of FIG. 12, a retrofit solution for the portion at the needle arrangement 11. But details concerning the realization of the cap 14 and of the electrical contacts are different.

FIG. 20 shows a perspective view of the catheter or cannula arrangement 1 with partially removed needle 110 and, accordingly, with open electrical contact. FIG. 21 shows a perspective view of the catheter or cannula arrangement 1 of FIG. 20 with inserted needle 110 and, accordingly, with closed electrical contact. The constituents have been described above, and corresponding reference numerals have been applied to corresponding parts.

As to protrusion or enlargement 175 of handle 17, also in this embodiment, it may allow attachment 19 to slide over handle 17 including protrusion or enlargement 175, so as to establish a firm fixing of attachment 19 to handle 17, e.g., as illustrated, in form of a snap-fit.

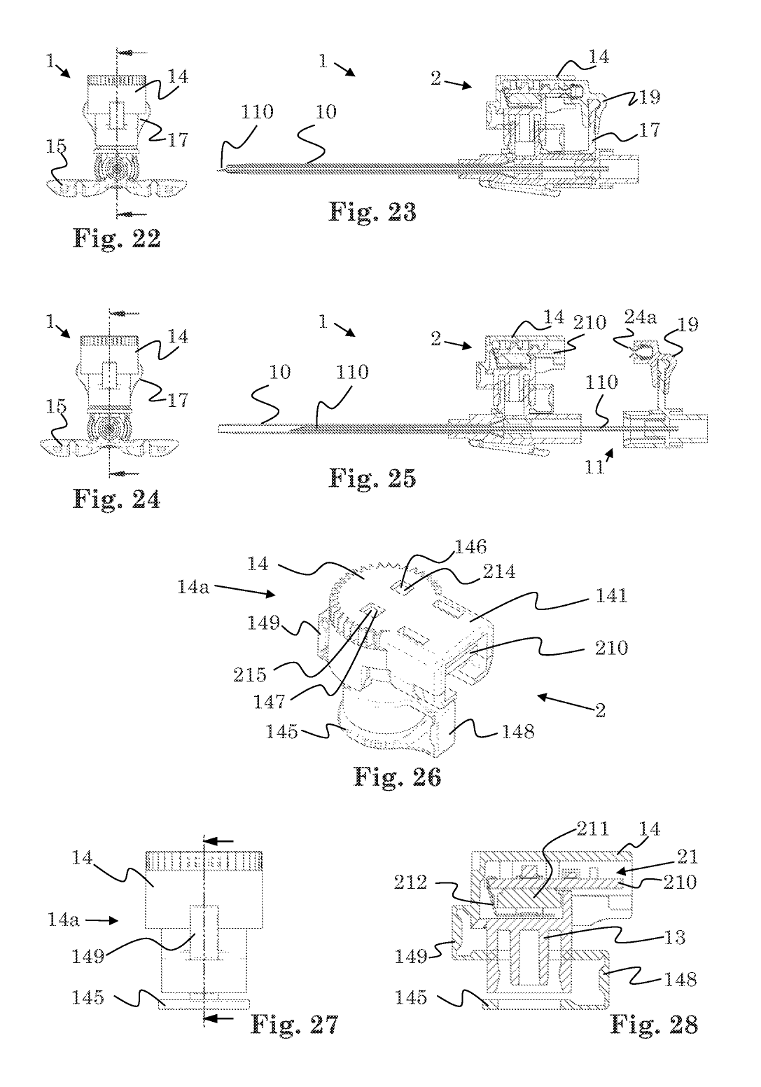

FIG. 22 shows a rear view in axial direction of the catheter or cannula arrangement 1 of FIG. 20 with inserted needle, the axial direction being given by the direction of elongation of catheter or cannula 10, a radial direction being perpendicular to the axial direction. FIG. 23 shows a cross-sectional view of the catheter or cannula arrangement of FIG. 20 with inserted needle, more particularly an axial cross-sectional view, the cross-section being taken as indicated in FIG. 22 by the dash-dotted line and the arrows.

FIGS. 24 and 25 illustrate the catheter or cannula arrangement 1 of FIG. 20 in the same way as FIGS. 22 and 23 do, but with a partially extracted needle 10.

FIG. 26 shows a perspective view of the cap 14 of the catheter or cannula arrangement 1 of FIG. 20 including a portion of a security unit 2. Cap 14 is formed by a member 14a which also includes loop-shaped cap fastener 145 and two interconnecting parts 148, 149. Member 14a and, more precisely, cap 14 has a protruding portion 141 and houses a PCBA including a PCB 210. LEDs 214, 215 or at least regions 146, 147 of cap 14 through which their light shall escape from cap 14 are also visible, too, in FIG. 26.

FIG. 27 shows a rear view of the cap 14 of the catheter or cannula arrangement 1 of FIG. 20. FIG. 28 shows a cross-sectional view of FIG. 26 including a portion of a security unit 2, the cross-section being taken as indicated in FIG. 27 by the dash-dotted line and the arrows. Member 14a forms also plug 13 for sealing injection port 12 (cf. FIG. 20). PCBA 21 includes, mounted on PCB 210, battery contact 212 and battery 211.

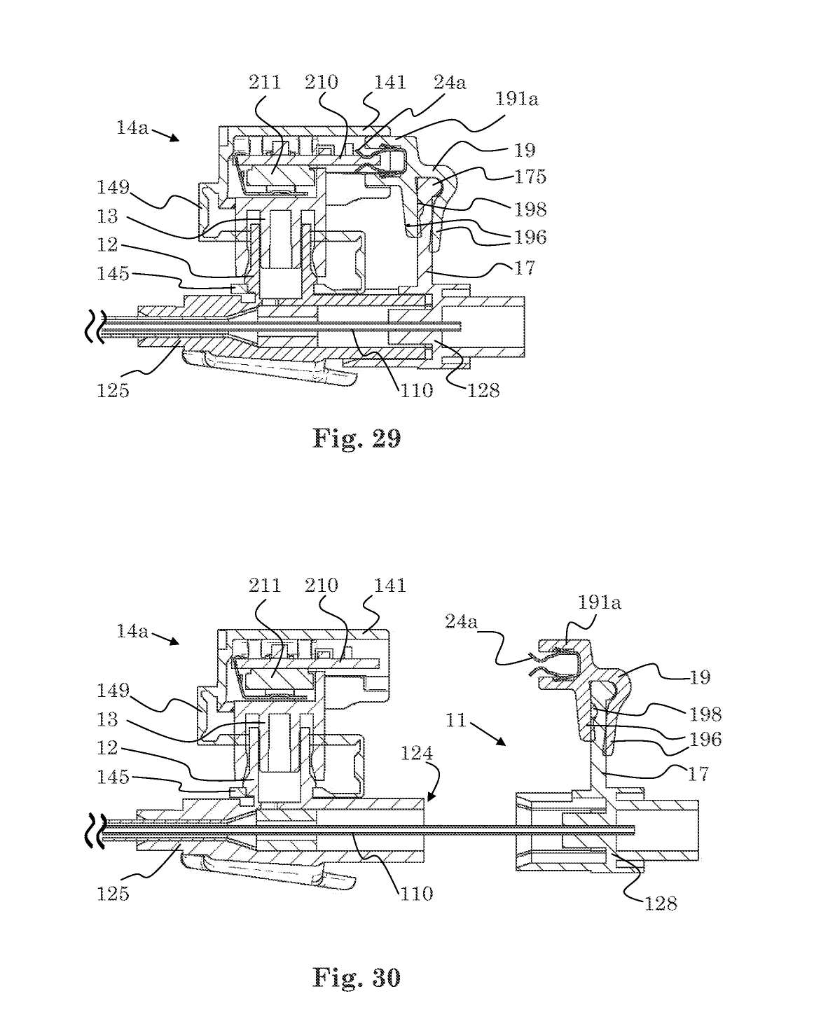

FIGS. 29 and 30 show enlarged details of the cross-sectional views of FIGS. 23 and 25, respectively. With needle 110 inserted (FIG. 29), electrical contact element 24a is slid onto PCB 210, so as to provide an electrical connection between at least two contact leads or printed circuit board tracks of PCB 210. The contact leads (cf. also FIGS. 15 and 17 for similar ones) may be on the same or on opposite sides of PCB 210, and when at least three, e.g., four, are provided, at least two may be on one and at least one on the other side. As described above, opening this electrical connection can be used for starting the time measurement.

Electrical contact element 24a (cf. FIG. 52 for a more detailed view) may be, as illustrated, a substantially U-shaped piece of sheet metal forming a clamp or jaws with tapering entrance, for providing a safe mechanical contact with PCB 210 and facilitating sliding it onto PCB 210. Electrical contact element 24a is a clamping contact element. Electrical contact element 24a itself is clamped between a pair of arms 191a of attachment 19 to handle 17 of needle arrangement 11 (cf. FIGS. 53, 54 for a more detailed view). Arms 191a form a protruding portion. Attachment 19 has a groove or channel 195 formed by a further pair of arms 196 at right angles to the pair 191a, to receive the handle 17. A protrusion 198 is provided on the inner arm 196 which interacts with a recess in handle 17 (not clearly visible in FIGS. 29, 30), for providing a snap fit and/or for defining and/or stabilizing a position of attachment 19 when attached to handle 17. Of course, alternatively or in addition, one could provide a recess or opening in attachment 19 and a corresponding protrusion at handle 17.

Protruding portion 141 and arms 191a are designed such that arms 191a are insertable in protruding portion 141. This may effect a (mechanical) guidance when moving needle arrangement 11 with respect to catheter or cannula 10, and it may provide a mechanical protection for the contact formed by electrical contact element 24a and contact elements present on PCB 210 (when needle 110 is inserted), more particularly mechanical protection versus bending or shear movements or versus radial or axial forces.

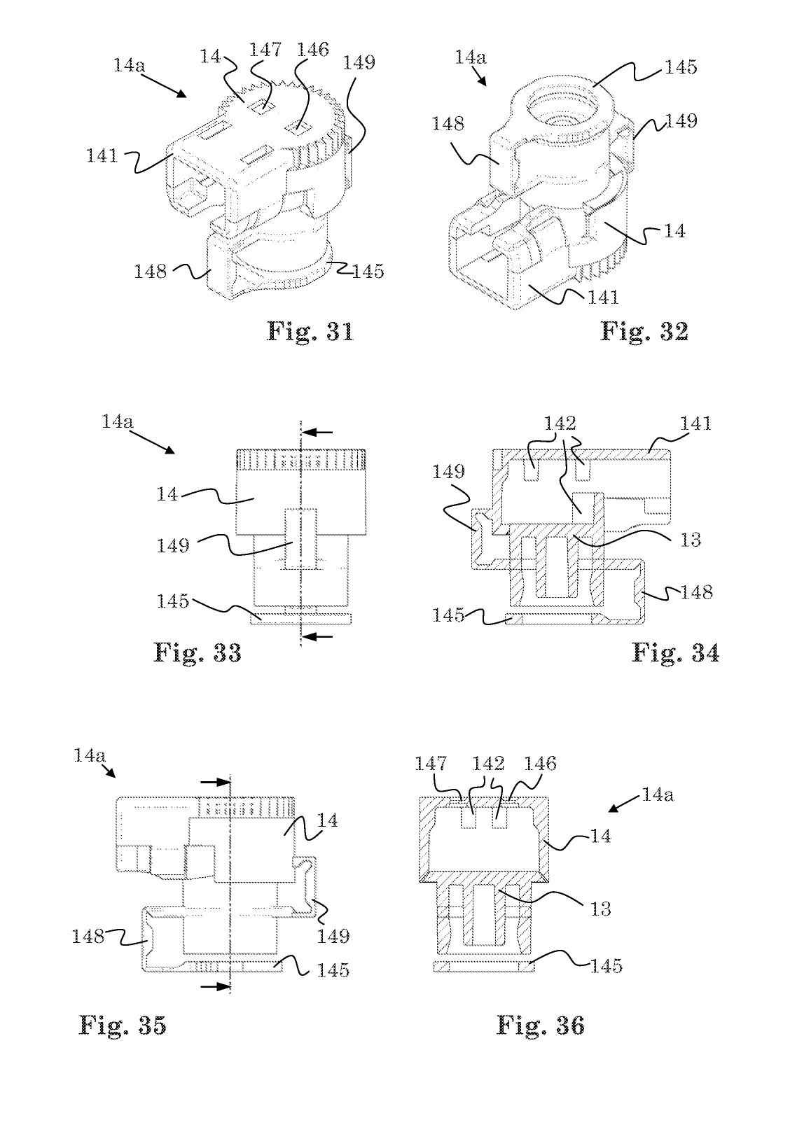

FIGS. 31 and 32 show, in top and bottom perspective views, the member 14a forming the cap 14 of the catheter or cannula arrangement of FIG. 20. If items 146, 147 are not openings, they may be transparent by being particularly thin (relative to adjacent portions of cap 14), cf. FIG. 36.

FIG. 33 shows a rear view (in axial direction) of the member 14a of the catheter or cannula arrangement of FIG. 20. FIG. 34 shows an axial cross-sectional view of the member 14, the cross-section being taken as indicated in FIG. 33 by the dash-dotted line and the arrows. Protrusions 142 are visible in FIG. 34 by means of which PCB 210 is clamped or fixed in position in cap 14. By protrusions 142, PCB 210 is mechanically contacted from opposing sides.

FIG. 35 shows a rear view (in radial direction) of the member 14a. FIG. 36 shows a radial cross-sectional view of the member 14a, the cross-section being taken as indicated in FIG. 35 by the dash-dotted line and the arrows.

The illustrated member 14a is, e.g., manufactured as a unitary or integrally formed part, in particular as a single injection molded part. It may fulfill a number of functions such as to make possible a simple insertion of PCBA 21 and to fix PCBA 21 in position (by protrusions 142). Member 14a is foldable.

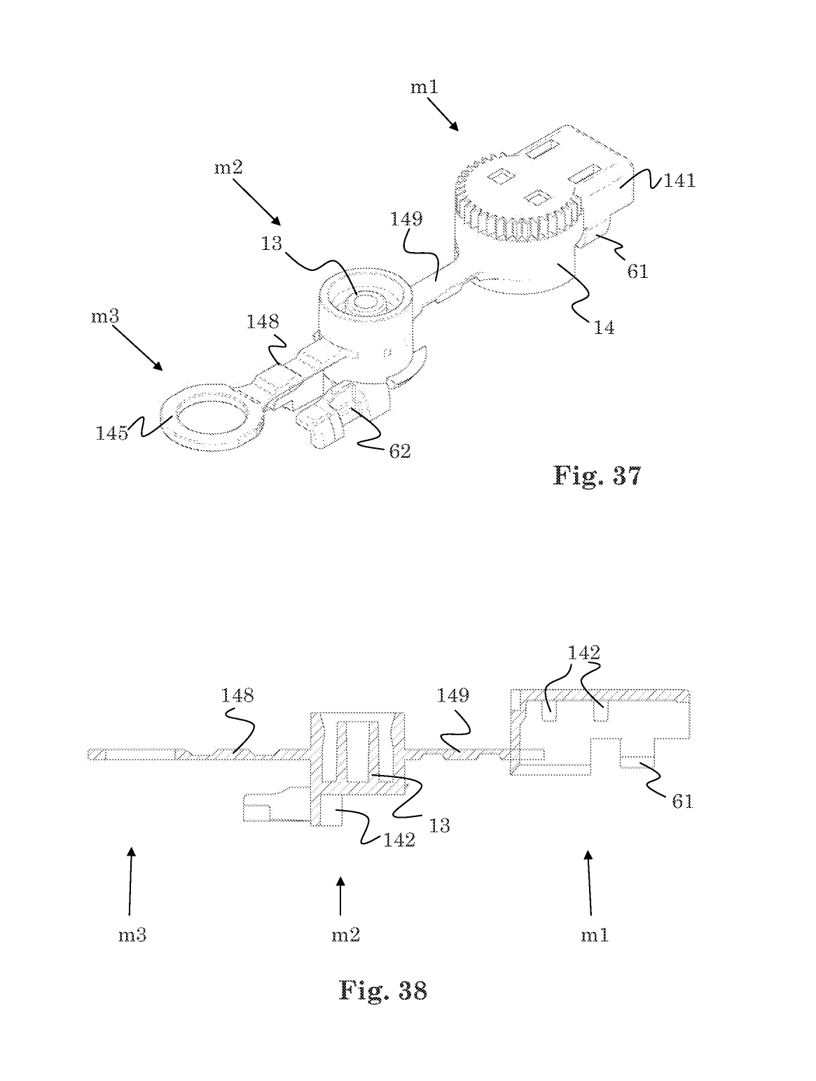

FIGS. 37 and 38 show in a perspective view and a cross-sectional view, respectively, the member 14a in unfolded state. Member 14a includes three main parts m1, m2, m3 which are pair-wise interconnected by interconnecting portions 148, 149, which form hinges allowing the member 14a to be folded into the configuration shown in FIG. 34.

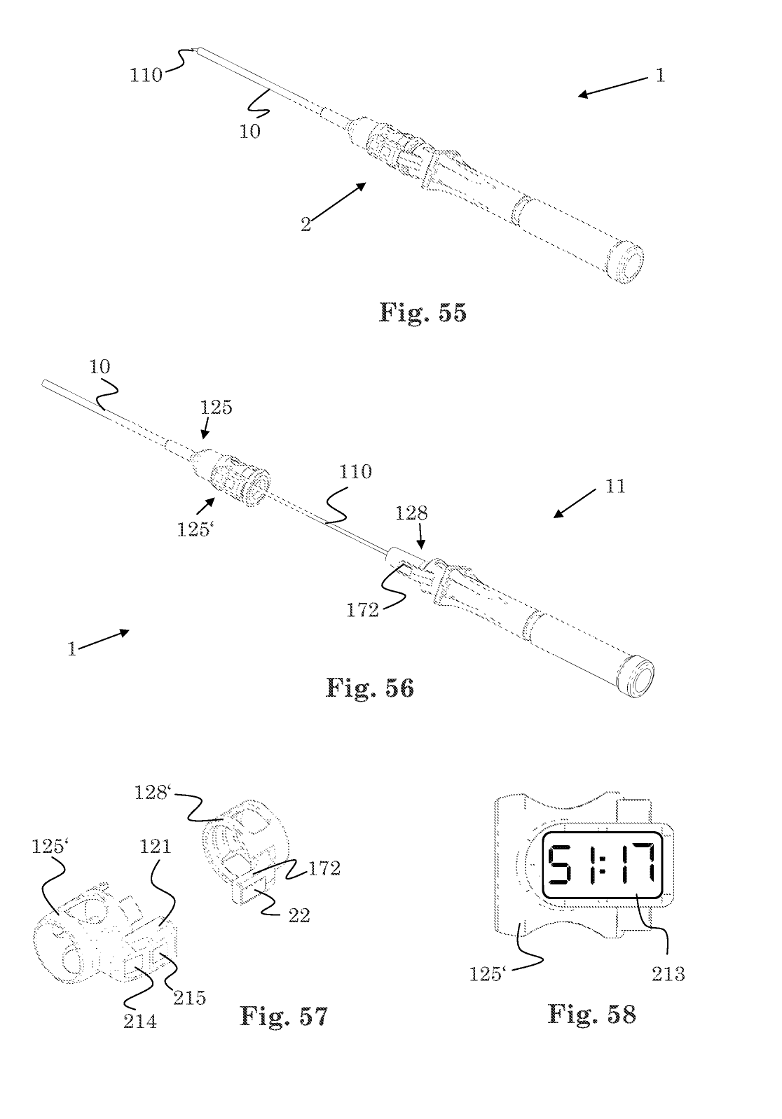

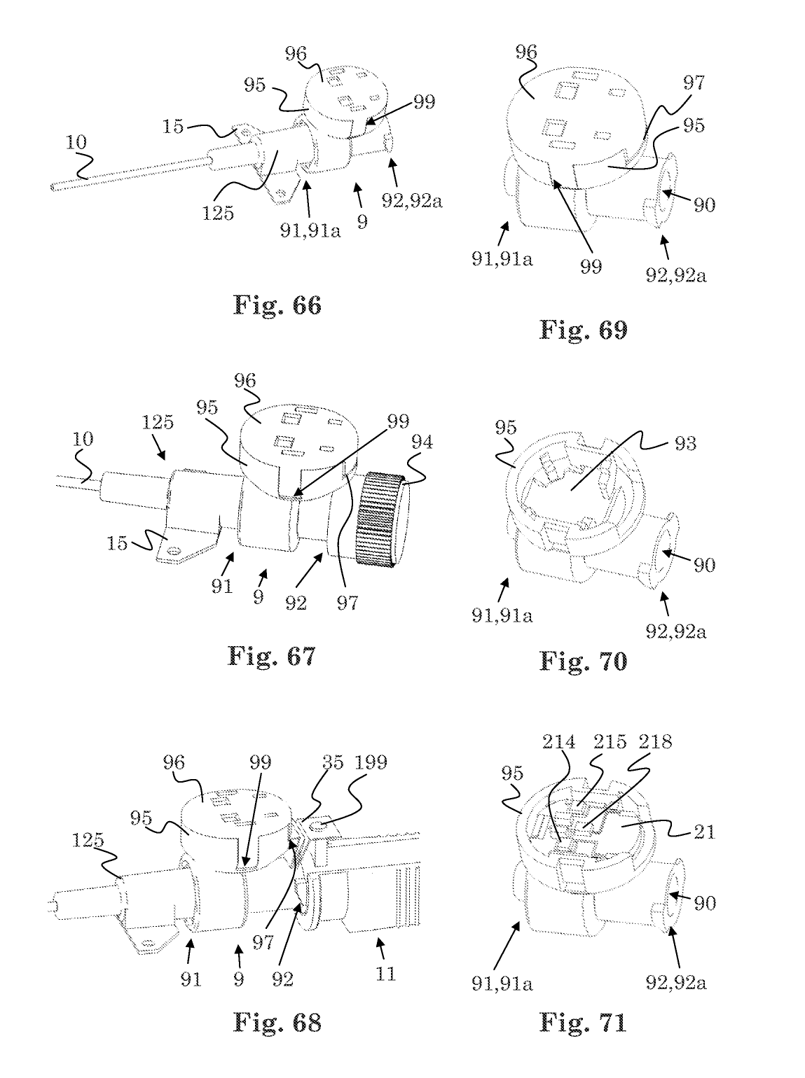

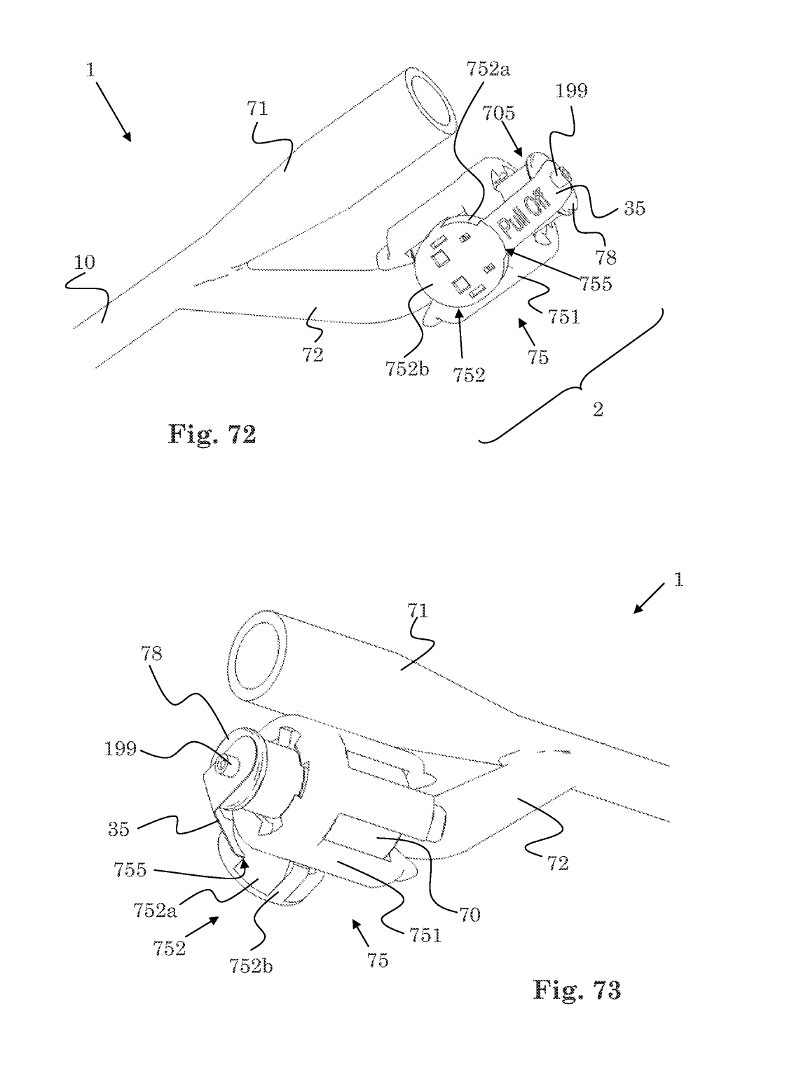

First part m1 forms a portion of cap 14 and forms a snap fit with second portion m2 forming a portion of cap 14 and plug 13. The snap fit is realized using outer part 61 of part m1 forming an arm with a protrusion and inner part 62 of part m2 forming an edge under which the protrusion can snap. Third part m3 forms a loop for attaching member 14a to the injection port of catheter or cannula arrangement 1. Note that, in general, i.e. not referring to a specific embodiment, a security unit 2 does not necessarily have to be (partly) arranged in a cap or in an attachment to a cap, and there is also no absolute need to have an injection port or to attach a portion of the security unit to the injection port or to an item attachable thereto. For example, a housing housing (at least) a portion of the security unit might be attached to or integrated in a holder 125 (cf. FIGS. 29, 30) holding the catheter or cannula 10. Such a holder 125 can be, e.g., a polymer body, e.g., manufactured using injection molding. Cf. FIGS. 55-57 below for corresponding embodiments. Or a housing may also be included in or provided by an extension of the catheter or cannula arrangement, e.g., cf. FIGS. 66 to 71 below. Or a housing may also be included in or provided by an attachment to a valve device, cf. FIGS. 72 to 76 below. Or a housing may also be included in or provided by and attachment to tubing of the catheter or cannula arrangement, cf. FIGS. 77 to 80 below.

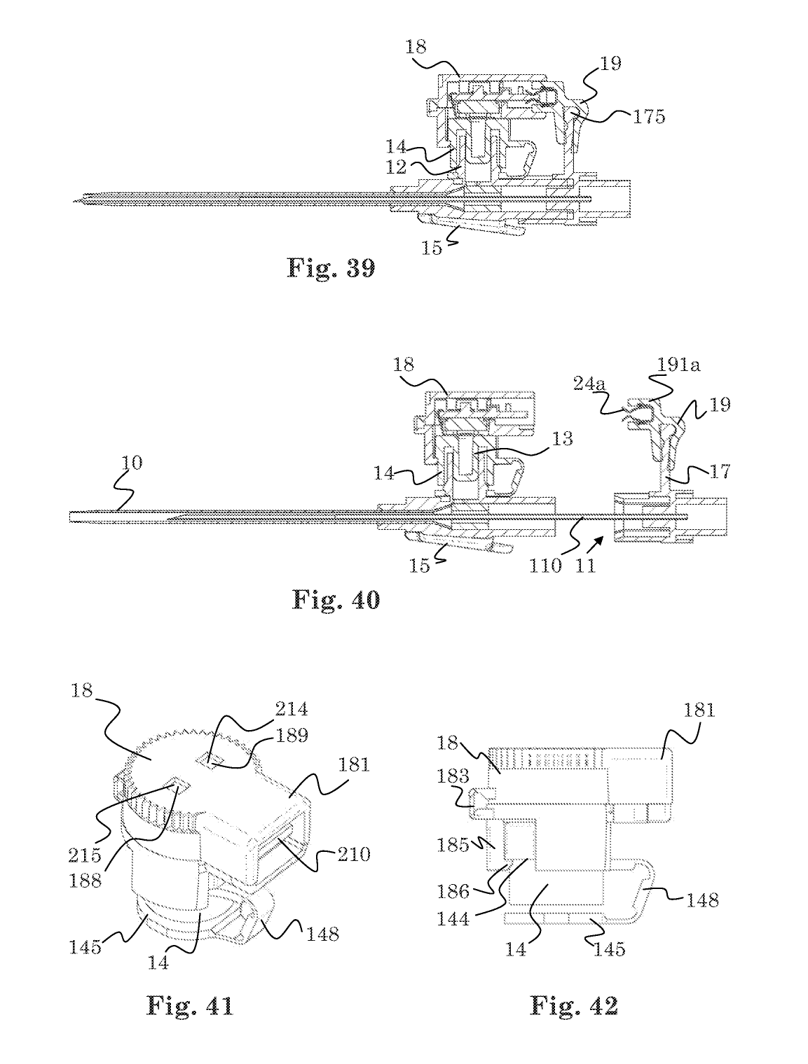

FIGS. 39 to 51 illustrate another catheter or cannula arrangement 1 which is in many functional aspects similar to the before-described ones, and it realizes, like the one of FIG. 12, a retrofit solution for attaching a portion of the security unit to the cap 14, and, like the one of FIG. 12 or of FIG. 20, a retrofit solution for the portion at the needle arrangement 11, wherein the realization of the electrical contacts is the same as described for the embodiment of FIG. 20. But details concerning the realization of the attachment 18 to the cap 14 (and thus of a corresponding member) are different. Thus, for the embodiment of FIG. 39, it is possible to start from a prior art standard catheter or cannula arrangement and attach thereto a security unit, so as to obtain an improved catheter or cannula arrangement.

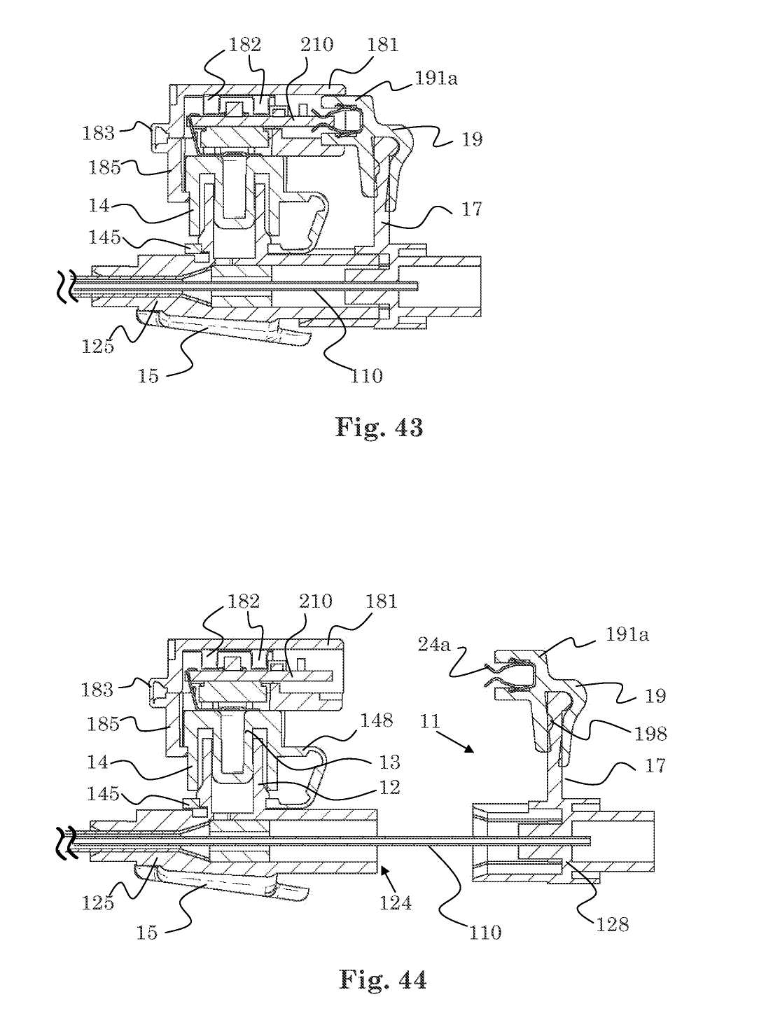

Note that, of course, it would also be possible to provide an integrated solution also for the portion at the needle arrangement 11 in this case, e.g., attachment 19 and handle 17 (cf. FIG. 43) forming a unitary part.

FIG. 39 shows a cross-sectional view of the catheter or cannula arrangement 1 with inserted needle 10. FIG. 40 shows a cross-sectional view of the catheter or cannula arrangement of FIG. 39 with partially removed needle 10.

FIG. 41 shows a perspective view of the cap 14 and attachment 18 to the cap of the catheter or cannula arrangement of FIG. 39, the attachment including a portion of a security unit 2. FIG. 42 shows a radial side view of cap 14 and attachment 18 of FIG. 41.

Attachment 18 has a snap fit connection with cap 14. More particularly, legs 185 of attachment 18 having a protrusion 186 can snap under an edge 144 of cap 14, so as to establish a snap fit attaching attachment 18 to cap 14.

FIGS. 43 and 44 show enlarged details of the cross-sectional views of FIGS. 39 and 40, respectively. With needle 110 inserted (FIG. 43), electrical contact element 24a is slid onto PCB 210, so as to provide an electrical connection between at least two contact leads or printed circuit board tracks of PCB 210. As described above, opening this electrical connection can be used for starting the time measurement.

Protruding portions 181 and 191a interact as described above for protruding portions 141 and 191a (cf. FIGS. 29, 30).

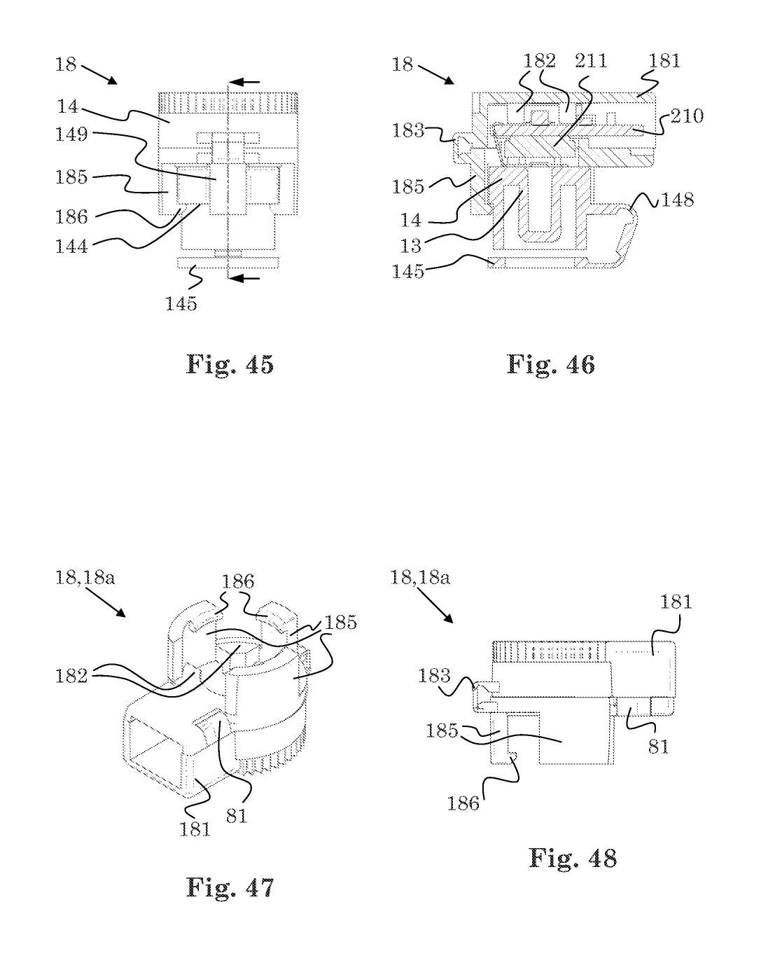

FIG. 45 shows a rear view of the cap 14 and attachment 18 to the cap of the catheter or cannula arrangement 1 of FIG. 39. FIG. 46 shows a cross-sectional view of the cap 14 and attachment 18 to the cap of the catheter or cannula arrangement 1 of FIG. 39 including a portion of a security unit 2, the cross-section being taken as indicated in FIG. 45 by the dash-dotted line and the arrows.

FIG. 47 shows a perspective view of the attachment 18 to the cap 14 of the catheter or cannula arrangement of FIG. 39. FIG. 48 shows a radial side view of this attachment 18 to the cap.

Attachment 18 to cap 14 may be formed by a member 18a which may be manufactured as a unitary or integrally formed part, in particular as a single injection molding part, similar to member 14a described above. It may fulfill a number of functions such as to make possible a simple insertion of PCBA 21 and to fix PCBA 21 in position (by protrusions 182). Member 18a may in particular be foldable.

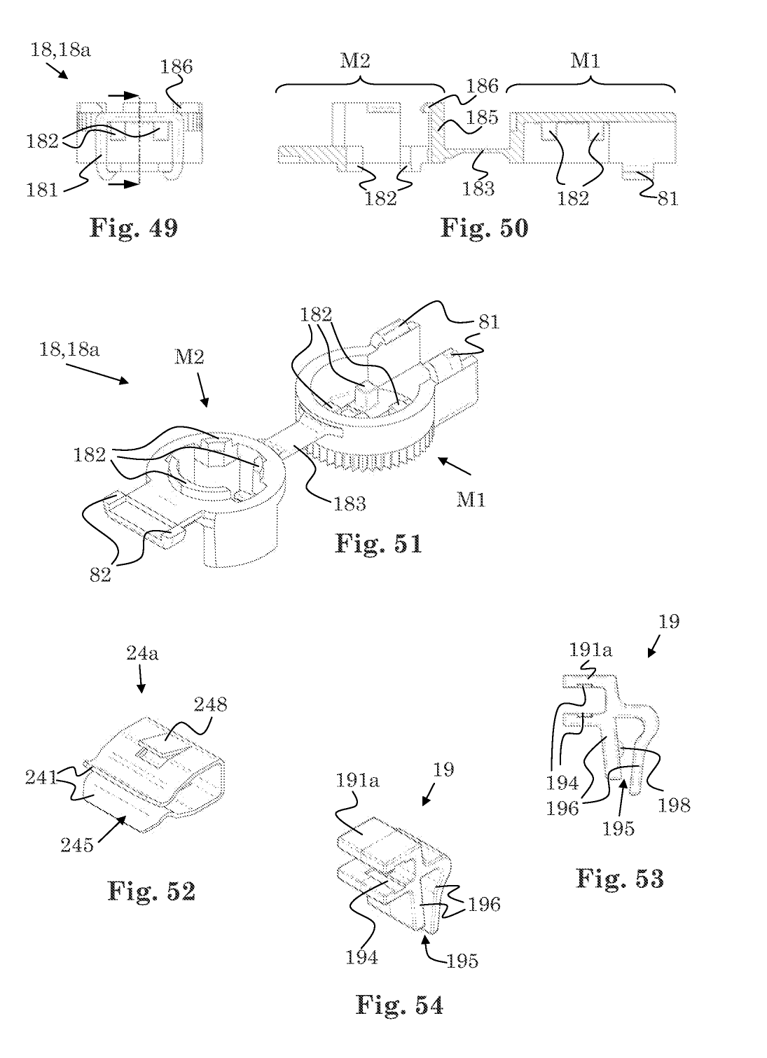

FIGS. 49 and 50 show an axial end view and an axial cross-sectional view, respectively, of the member 18a forming the attachment to the cap of the catheter or cannula arrangement of FIG. 39 in unfolded state. Member 18a includes two main parts M1, M2, which are interconnected by an interconnecting hinge portion 183. Parts M1, M2 are fixable to each other by a snap fit, e.g., formed by outer parts 81 and inner parts 82, similar to what is described above for first and second parts m1, m2.

FIG. 51 shows a perspective view of the member 18a in unfolded state. Protrusions 182 for holding PCBA 21 in place are clearly shown in FIG. 51.

FIG. 52 shows a perspective view of the electrical contact element 24a of the security unit 2 of the catheter or cannula arrangement 1 of FIG. 20 or of FIG. 39. It is a piece of sheet metal bent to form a tapering entrance 245 formed by contact jaws 241. Catches 248 are formed in element 24a (e.g., by punching) for ensuring that it is not pushed out of attachment 19 to handle 17 when it is slid off PCB 210 when needle 110 is removed from catheter or cannula 10. For this purpose, catches 248 cooperate with recesses 194 (cf. FIGS. 53, 54) of attachment 19 to handle 17 of needle arrangement 11.

FIGS. 53 and 54 are cross-sectional and perspective views, respectively, of the attachment 19 to the handle 17 of the catheter or cannula arrangement 1 of FIG. 20 or of FIG. 39. An electrical contact element such as item 24a of FIG. 52 may be inserted therein. The features and functions of such an attachment 19 have already been described above.

FIG. 55 is a perspective view of another catheter or cannula arrangement 1, in an initial state with needle 110 inserted in catheter or cannula 10. FIG. 56 shows in the same manner the catheter or cannula arrangement 1 of FIG. 55, but with needle 110 partially removed out of catheter or cannula 10. Most of the constituents have been described above, and corresponding reference numerals have been applied to corresponding parts.

In this embodiment, the catheter or cannula has neither an injection port nor a handle of the needle arrangement 11. This embodiment illustrates that it is possible to arrange components of the security unit 2 elsewhere in the absence of these components. Of course, any combination of locations of security unit components staying with the catheter or cannula and of security unit components staying with the needle arrangement are possible, in particular as far as locations are described in the present patent application, such as at or in a cap or at or in an attachment to a cap or at or in a shaft part for components staying with the catheter or cannula, and at or in a handle or at or in an attachment to a handle or at or in a shaft part for components staying with the needle arrangement.

FIG. 57 is a perspective view of constituents of the catheter or cannula arrangement 1 of FIGS. 55, 56. More particularly, it illustrates the security unit integrated in two shaft parts or, more precisely, two attachments 125′, 128′ to shaft parts, namely to shaft part 125 holding catheter or cannula 1 (also referred to as holder 125) and to shaft part 128 holding needle 110 (also referred to as holder 128). Of course, instead of having the security unit integrated in the attachments 125′, 128′ (which makes possible to retrofit known or standard catheter or cannula arrangements), one could also integrate it directly into the respective shaft parts or holders 125, 128. The functionality of this embodiment may substantially coincide with what has been described above for other embodiments. In particular, two light emitters 214, 215 may be provided for signalling the status, and on a protruding portion 172 of the needle arrangement, an electrical contact element 22 such as a metal coating may be provided, for bridging (in the state illustrated in FIG. 55) two electrical contacts of the security unit which are located inside a protrusion 121 of shaft part 125 but not visible in FIGS. 55-57.

FIG. 66 is a perspective view of a catheter or cannula arrangement 1 including an extension 9. The functionality of this embodiment may substantially coincide with what has been described above for other embodiments but extension 9 provides an alternative for locating at least a portion of the security unit and makes possible to retrofit existing catheters or cannulas. Extension 9 is connected, at its first end 91, to the connection end (not actually visible in FIG. 66) of the catheter or cannula arrangement 1, the connection end being provided by shaft part or holder 125 for holding the catheter or cannula which may be integrally formed with or be a part distinct from the catheter or cannula. The first end 91 provides a connector such as a plug 91a. The second end 92 of the extension 9 provides a connector 92a which, for example, replicates the interconnection properties of the connecting end. Thus, devices such as syringes or tubes for taking up fluids flowing through the catheter or cannula 10 or for supplying fluids (to the body) via the catheter or cannula can be connected to the catheter or cannula arrangement 1, in particular in the same way as without the extension (only in a displaced location).

The extension 9 can locate or house at least a portion of a security unit such as a printed circuit board assembly. For accomplishing this, extension 9 includes, in the embodiment of FIG. 66, a first housing part 95 and a second housing part 96 which may, e.g., form a snap fit 99. Extension 9 including housing parts 95 and 96 can, e.g., be an integrally formed part; or, as illustrated in FIG. 66, housing part 96 is a part distinct from housing part 95 and separately manufactured.

FIG. 67 is a perspective view of a detail of the catheter or cannula arrangement 1 of FIG. 66, further including a closure 94 such as a cap attached to the connection end.

FIG. 68 is a perspective view of a detail of the catheter or cannula arrangement 1 of FIG. 66, but including a needle arrangement 11. Shaft part or holder 125 and needle arrangement 11 are mutually interconnected by extension 9. The needle arrangement 11 may include, as illustrated, a needle protection for avoiding access to the needle during and after the removal of the needle from the catheter or cannula 10.

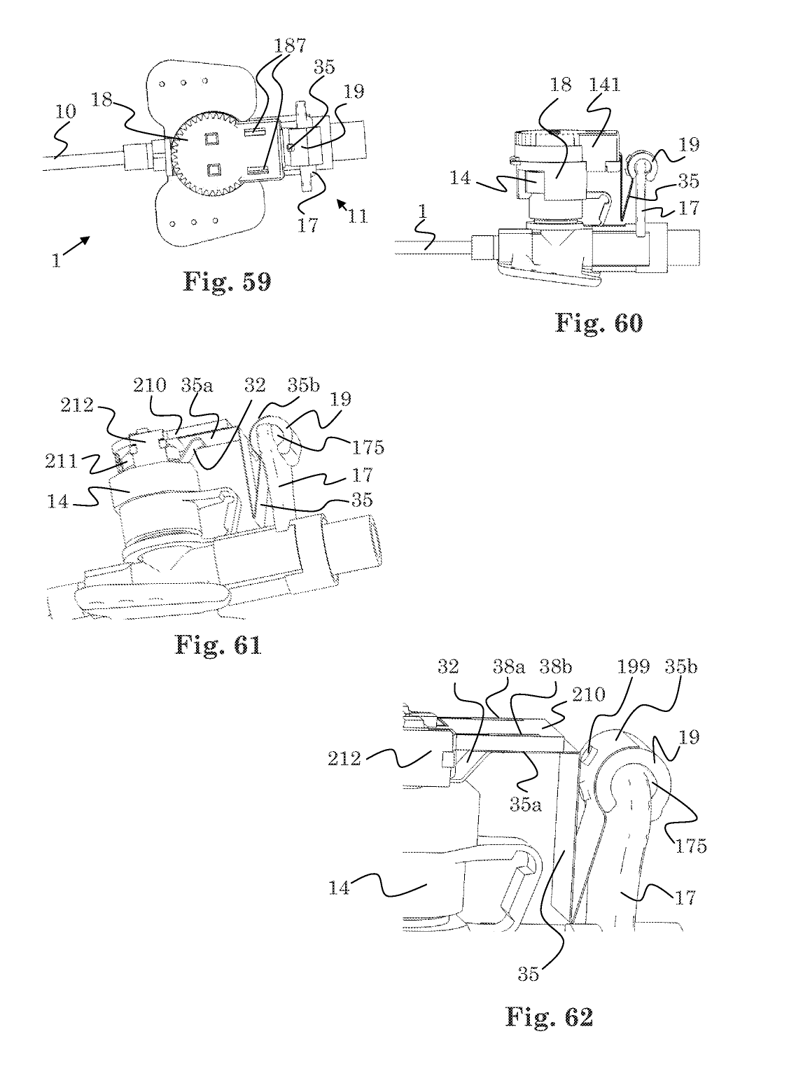

Furthermore, security unit 2 in full is present in the catheter or cannula arrangement of FIG. 68. A separating element 35 fixed at a protrusion 199 of the needle arrangement 11 is present which interacts with further parts of the security unit 2 present inside the housing provided by housing parts 95 and 96 in a way described elsewhere in the present patent application, cf., e.g., FIGS. 59 to 65. Separating element 35 extends through an opening 97 provided between housing parts 95 and 96, wherein opening 97 can be a slit, as illustrated in FIGS. 67, 68 and 69 below. Of course, also other ways of starting a dwell time measurement are possible when using an extension.

FIG. 69 is a perspective view of the extension 9 of FIGS. 66 to 68. An inner channel 90 provided by extension 9 is visible and indicated. A fluid can flow through the inner channel 90 from the first end 91 to the second end 92 or vice versa.

FIG. 70 is a perspective view of the extension of FIGS. 66 to 69, but with housing part 96 removed. An inner volume 93 provided by housing parts 95, 96 is therefore visible in FIG. 70. A portion of the security unit can be accommodated in the inner volume 93.

FIG. 71 is perspective view of the extension of FIG. 70 but with an inserted printed circuit board assembly 21 of the security unit. Design and function of that portion and other portions of the security unit may be as described elsewhere in the present patent application.

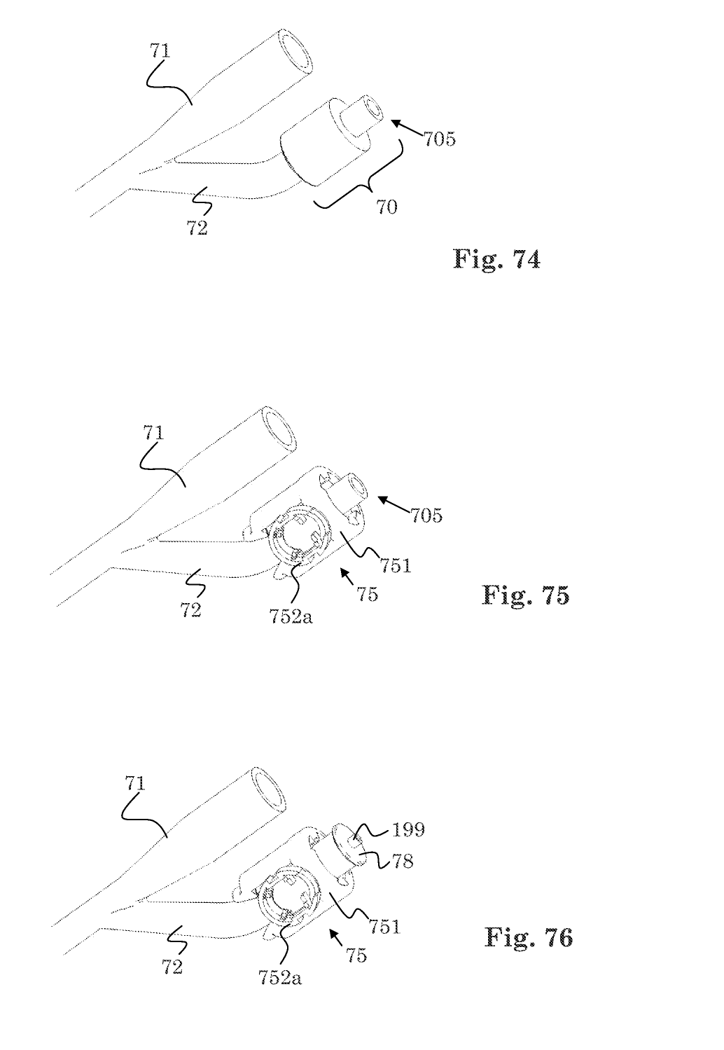

FIGS. 72 and 73 are perspective views of a detail of a catheter or cannula arrangement 1 including a valve device 70 to which a security unit 2 is connected or more particularly fastened. FIG. 74 is a perspective view of a detail of the catheter or cannula arrangement 1 of FIGS. 72 and 73 with all parts of the security unit 2 removed. FIG. 74 may be interpreted as illustrating a detail of an existing urinary catheter or more particularly of a known Foley catheter. FIG. 75 is a perspective view of a detail of the catheter or cannula arrangement 1 of FIG. 74 with an attachment 75 to the valve device 70 attached. In the perspective view of FIG. 76, in addition, an access inhibitor, e.g., embodied as a closure 78 (which also constitutes an attachment to the valve device) is attached to an access end 705 of the valve device 70. FIGS. 74 to 76 are merely to illustrate the valve device 70 and design and arrangement of the security unit at the valve device 70.

The catheter or cannula arrangement 1 of FIGS. 72, 73 provides two separate channels 71 and 72. At the end of second channel 72, valve device 70 is present, cf. FIG. 74. The valve device 70 has an access end 705 for accessing the valve of valve device 70 (the valve itself is not visible in the Figures). The Figures illustrate a portion of the catheter or cannula arrangement 1 close to an outer end thereof. The inner end, which is that end which is meant to be present inside a body, is not shown.

At the inner end, a balloon may be present which can be expanded by introduction of a fluid via the second channel 72. Such a balloon can be provided for holding the catheter in place (inside the body). Valve device 70, and more precisely the corresponding valve, makes possible to selectably enable or inhibit fluid getting into or out of the second channel (and the balloon).

Two items are attached to valve device 70: attachment 75 to valve device 70 and closure 78 (which may also be considered a cover or more generally an access inhibitor) such as a cap or a plug. Attachment 75 may form a snap fit with valve device 70, as illustrated, cf., e.g., FIG. 73. In the illustrated embodiment, attachment 75 includes a first part 751 and a second part 752, which are interconnected, wherein part 752 provides a housing and includes a first portion 752a and a second portion 752b which may be distinct parts or may, in particular, form a single integrally formed (e.g., injection molded) part. Portion 752a may form a single integrally formed (e.g., injection molded) part with first part 751. First part 751 surrounds or encompasses the valve device 70, at least in part.

Part 752 locates or houses a portion of the security unit, in particular a printed circuit board assembly, which is operationally connected to closure 78, e.g., as illustrated, via a strip and more particularly a separating element 35. Separating element 35 is fixed to closure 78, e.g., via a protrusion 199, and extends through an opening 755 provided between portions 752a and 752b, wherein opening 755 can be a slit, as illustrated in FIGS. 72, 73. Closure 78, separating element 35 and at least a portion of the portion of the security unit located in part 752 can be considered as constituents of an activation detection unit, cf. above for possible functions and functions of activation detection units.

In a typical use situation for the described catheter or cannula arrangement with a valve device 70, approximately at the time of starting to use the catheter, e.g., briefly after inserting it into the body, access to the access end 705 is required. In order to achieve this access, cover or closure 78 has to be removed from access end 705. However, the security unit 2 and more particularly closure 78 and separating element 35 are structured and arranged such that an (ordinary) removal of closure 78 from the access end 705 causes separation element 35 to move, at least in part, out of part 752, so as to let a formerly open electrical contact close which again can cause the dwell time measurement to start, cf., e.g., the more detailed description of FIGS. 59 to 65 and of other descriptions of activation detection units for more details. Further modifications and possibilities are obvious in view of the disclosure of the present patent application. For example, instead of closing a contact when removing closure 78 (and a separating element attached thereto), an electrical contact could be opened instead of closed, cf., e.g., the embodiments of FIGS. 7 and 17. Or, it could also be provided that the valve device 70 itself is already structured and arranged to locate or house at least a portion of the security unit 2, instead of the attachment 75 to the valve device 70 doing so. However, using an attachment 75 to the valve device makes possible to retrofit existing catheters such as standard urinary and/or Foley catheters.

So far, mainly embodiments have been described in more detail which signal the status but do not display the dwell time. The latter can, however, be realized, e.g., as illustrated in FIG. 58.

FIG. 58 is an illustration of a portion of another catheter or cannula arrangement, with a time display 213. Such a time display 213 (usually a portion of the signalling unit) may be provided in any of the other described embodiments, either replacing other signalling means such as signal emitters 214, 215, or in addition thereto. It may display, e.g., the dwell time (as inferred using the timer or time piece), or the time remaining until a preset time limit (such as 72 hours from insertion) will have expired.

FIG. 58 may also be understood as an illustration of a portion of an embodiment corresponding to the one of FIGS. 55-57, wherein the signal emitters 214, 215 are replaced by time display 213. Time display 213 may be realized, e.g., as a liquid crystal display. However, a time display 213 may be also provided in other places and embodiments, e.g., in a cap or in an attachment to a cap (cf. the various above- and below-described embodiments).

In conjunction with FIGS. 77 to 80, another, special aspect of the invention will be described in which a display, in particular a numeric or alphanumeric or graphic display, e.g. an LCD display, can be useful. This special aspect concerns catheter or cannula arrangements including a sensing element such as a temperature sensing element.

FIG. 77 is a perspective view of a detail of a catheter or cannula arrangement already known in the art which includes a sensing element. Basically, an outer end 102 of the catheter or cannula arrangement is shown. The present description of the special aspect of the invention uses an example of a urinary catheter with temperature sensing, more particularly of a Foley catheter with a temperature sensing element. However, the special aspect is not bound to a specific kind of catheter (or catheter or cannula arrangement) and, moreover, in a general view, this special aspect does not even require the presence of a security unit.

In FIG. 77, the temperature sensing element itself is not visible, but the catheter or cannula arrangement has a sensing channel 73, which in the illustrated example is a third sensing channel in addition to a first channel 71 and a second channel 72. The three channels are separate from each other, wherein first and second channels 71, 72 are channels for guiding fluids, whereas the sensing channel 73 is an electrical channel, usually substantially provided by an electrically insulated conductor such as an insulated electric cable.

The cable 732 has an electrical connector 731 at its (outer) end by means of which an external reading device can be connected to the sensing element.

Channels 71 and 72 can be identical to those described for FIG. 74, cf. there for details.

FIG. 78 is a perspective view of a catheter or cannula arrangement 1 including an electronics unit 74. This catheter or cannula arrangement can be obtained by retrofitting the catheter or cannula arrangement of FIG. 77 with an electronics unit arrangement including an electronics unit 74.

Electronics unit 74 is present at outer end 102 (and not at inner end 101) and is operationally (and more precisely electrically) connected to the sensing element via cable 732 and connector 731. E.g., electronics unit 74 may include a socket for that purpose.

Electronics unit 74 includes an attachment to the catheter or cannula which can also be considered a housing 735, and (optionally) a display 738 and (optionally) a user control 739 such as a switch or button.

FIG. 79 is a perspective view approximately onto the front of a detail of a catheter or cannula arrangement which is largely identical to the one of FIG. 78, merely the display 738 and the user control 739 are different (mainly concerning their design only). FIG. 80 is a perspective view approximately onto the back of a detail of the catheter or cannula arrangement of FIG. 79. The catheter or cannula arrangements of FIGS. 78 to 80 are assumed to be functionally identical and will therefore be jointly described.

As can be seen from FIGS. 78 to 80, the described example is related to an embodiment as illustrated in FIGS. 72 to 76 in that a similar security unit and more particularly a similar activation detection unit is realized in both cases. (For details, it is also referred to the FIGS. 72 to 76 and the related description.) The main difference is the design of the housing 735 and of the attachment 75, respectively. The printed circuit board assembly and the electronic circuitry, respectively, is housed differently, and the corresponding housing (or attachment 75) is, in case of FIGS. 72 to 76 attached solely to the valve device 70, whereas in case of FIGS. 78 to 80, it is attached to tubing of all channels of the catheter. For the latter, fixing structures 734 are provided at housing 735 which even may be integrally formed as a single part with a portion of housing 735. These fixing structures 734 are clamping members, but other ways and structures for attaching housing 735 to the catheter are also possible.

However, as far as the activation detection unit is concerned, the same provisions are present in FIGS. 72 to 76 as in FIGS. 78 to 80, cf., e.g., closure or access inhibitor 78 at access end 705 of valve device 70, separating element 35 and a slit-like opening 755.

Thus, electronics unit 74 not only relates to a sensing element of the arrangement and thus can, e.g., make possible to monitor a temperature inside a body, but it also realizes essential functions of a security unit, thus allowing to monitor the dwell time and, e.g., provide an alarm in case a preselected maximum dwell time is reached.

Furthermore, the activation detection unit may also initiate the action of the electronics unit 74. Thus, the processing and/or storing accomplished in electronics unit 74 may be caused to start simultaneously with the dwell time measurement. For example, with separating element 35 in its initial position, a battery of the catheter or cannula arrangement is in unloaded state and thus, the catheter or cannula arrangement or at least the security unit and the electronics unit are not powered. With separating element 35 removed from its initial state, the battery is loaded and powers the security unit and the electronics unit. Such a provision can be very useful in particular if catheter or cannula arrangements are stored for some time, e.g., for months or years, before they are actually used, because during the time of storage before use, the energy consumption is zero or at least very low.

Display 738 can be, e.g, an liquid crystal display by means of which, e.g., a currently sensed temperature (as sensed by the sensing element) can be displayed. It may be provided that the display 738 continuously displays some data. However, it can also be provided that display 738 displays data only in reaction to a request or rather to a request signal. For example, operating user control 739 may produce a request signal. Another request signal may be produced by the electronics unit 74 in case a signal produced by the sensing element fulfills a predetermined condition, e.g., is above a threshold value, indicative of the sensed physical quantity exceeding a predetermined value. In that case, the display 738 might, e.g., display a sensed temperature and/or a warning (because an inflammation appears to be likely). And still another request signal may be produced by the security unit in case a preselected maximum dwell time is exceeded or in case a preselected maximum dwell time will be exceeded within a preselected time duration.

Displaying data only in reaction to a request or a request signal makes possible to save energy which otherwise would be wasted by display 738. This can be particularly important in case of catheters or cannulas with a relatively long standard dwell time such as urinary catheters.

Another way of saving energy is to provide not a graphical or numerical or alphanumerical display, but to use, e.g., one or more light emitting diodes (LEDs), such as the two light emitters 214, 215 described elsewhere in the present patent application, and to operate them in a pulsed fashion.

Electronics unit 74 includes electronic circuitry (not illustrated) for processing signals outputted by the sensing element, e.g., for amplifying them or converting them into digital signals. Alternatively or in addition, the electronic circuitry may be provided for obtaining data related to signals produced by the sensing element, e.g., so as to derive from the signals a sensed temperature in degrees Celsius. The electronic circuitry may alternatively or in addition also be provided for storing data related to signals produced by the sensing element. The electronic circuitry may be comprised, e.g., in a chip present on a printed circuit board.

In particular, such data related to signals produced by the sensing element may be stored repeatedly, in particular periodically. Corresponding data sets including data related to signals produced by the sensing element at different times may, e.g., be analyzed, either within the electronics unit or using an external device.

For transmitting data or data sets to an external device, the electronics unit may comprise, e.g., a wirebound interface such as a serial digital interface or a wireless transmission interface, e.g., a radio frequency transmitter or transceiver. It can also be provided that a wireless transmission is initiated (by the electronics unit) in reaction to a detection of a dangerous situation or of an emergency situation. For example, when a too high temperature is sensed and/or when a preselected maximum dwell time is exceeded, an alarm signal is (automatically) wirelessly transmitted. And/or it can be provided that a wireless transmission of data is accomplished upon a request, more particularly in reaction to a request signal, like described above for causing the display to display data.

It is also possible that a transmitter or transceiver, in particular a radio frequency transmitter or transceiver, is provided not only for transmitting data related to the dwell time and/or data related to the sensed physical magnitude, but also of identification data. The latter make it possible to identify the catheter or cannula or at least a component thereof. And thus, the data related to the dwell time and/or the data related to the sensed physical magnitude can be identifiable as originating from the identified catheter or cannula arrangement. The identification data are stored in the security unit and/or in the electronics unit, e.g., in the storage unit for storing data related to signals produced by the sensing element.

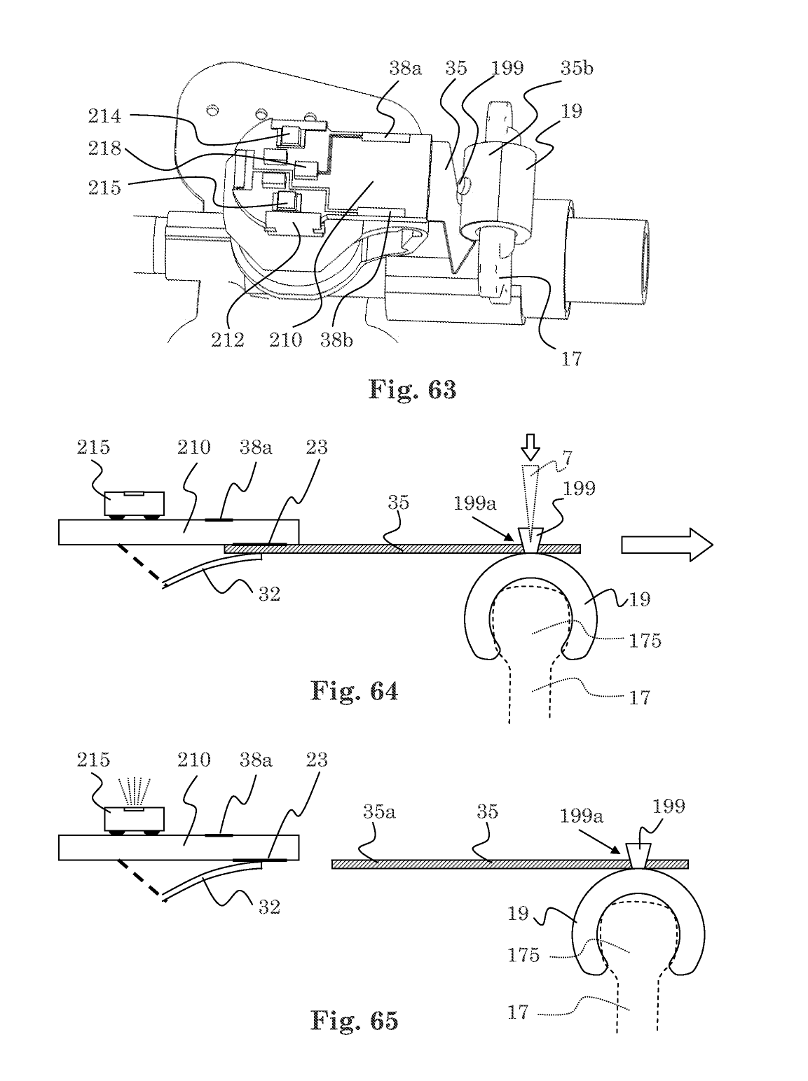

FIGS. 59 and 60 are top and side views, respectively, of a detail of a catheter or cannula arrangement 1 with pull-out tab or separating element 35, with inserted needle. FIG. 61 is a perspective view thereof with the attachment 18 to the cap 14 (more precisely the housing thereof) not shown, and FIG. 62 is a perspective view of an enlarged detail of the catheter or cannula arrangement of FIGS. 59 to 61, again with the housing portion of the attachment 18 to the cap 14 not shown, and FIG. 63 is another perspective view (approximate top view) thereof. FIG. 64 is a schematic illustration of a portion of a security unit with pull-out tab in an initial position, in a side view, FIG. 65 is a schematic illustration of the portion of the security unit of FIG. 64, with pulled-out pull-out tab 35, in a side view. The constituents shown in FIGS. 64, 65 can, together, be considered a part or an assembly, which may be manufactured separately from further parts of the catheter or cannula arrangement.

Many of the constituents shown in FIGS. 59 to 65 have been described above, wherein corresponding reference numerals have been applied to corresponding parts.

In this embodiment, pulling the needle out of the catheter or cannula 10 does not effect an opening of an electrical circuit, but a closing of an electrical circuit (or a bridging of electrical contact elements). In the state with inserted needle illustrated in FIGS. 59 to 64, a separating element 35 such as a polymer foil strip or another sheet-like insulating member is located between an electrical contact element 32, e.g., formed by battery contact 212, and another electric contact element 23, e.g., formed by printed circuit board 210 and more particularly by a contact pad thereof. The two electrical contact elements 23 and 32 form a spring-loaded contact, such that with separating element 35 removed from between them, they will form a closed electrical contact. In FIGS. 64, 65, the thick dashed line indicates an operational (more particularly: mechanical) connection between printed circuit board 210 and an end of contact element 32. The corresponding closing of an electrical contact or circuit can be detected by the security unit, e.g., by detecting the effected change in electrical resistance, similarly to what has been described above. In reaction to the detection, the timer may be started or the time measurement may be started. Initiating the time determination may in this case be effected by powering the electronics (in particular the integrated circuit 218) of the security unit, in other words by starting using (and thus draining) the battery. This can provide a greatly increased shelf life of the catheter or cannula, because after manufacture (usually including testing), no power needs to be drained from the battery 211 before the catheter or cannula is applied, i.e. inserted into a body. However, it is also possible to provide that after manufacture, the electronics of the security unit is and remains powered until after removal of the needle, and the removal of the separating element 35 is mainly used as an indication that the dwell time starts and thus that the timer has to start.

In order to save battery power—and thus in particular in the above-described case that removing the separating element 35 from between electrical contacts powers up the electronics of the security unit—it may be provided that testing (in particular after manufacture of the assembly illustrated in FIGS. 64, 65, and possibly after attaching the assembly to further components of the catheter or cannula arrangement) can be accomplished using two electrical contact elements (such as contact pads) of printed circuit board 210, such as contact elements 38a, 38b (cf. FIG. 62), e.g., accessing them through openings (or slits) 187 (cf. FIG. 59).

For mechanically connecting separating element 35 (at end 35b) to the needle arrangement or, more specifically, to the attachment 19 to handle 17 (indicated in FIGS. 64, 65 in dashed lines only, since it does not belong to the illustrated assembly), a provision (or protrusion) 199 is provided. Protrusion 199 is shaped such that it attaches a polymer foil 35 having a hole of suitable size fixedly to attachment 19. Protrusion 199 is initially substantially cylindrical, enabling the hole in the polymer foil 35 to be pushed over protrusion 199. Protrusion 199 can then be deformed so as to produce a desired waist 199a, e.g., by piercing a tip 7 of a tool into protrusion 199, thus locally widening the same, cf. FIG. 64, so that the foil cannot be removed. In the embodiment of FIGS. 59 to 63, two protrusions for fixing the second end 35b of separating member 35 are provided.

Starting the timer or the time measurement in reaction to the removal of the needle has advantages described further above. The second end 35b of separating element 35 may be fixed to the needle arrangement in various ways, e.g., by gluing it to the attachment 19 to handle 17 or directly to handle 17. Using, as described above and illustrated in FIGS. 59 to 65, a (mechanical) fixing element such as the attachment 19 to handle 17 (or an attachment to a shaft part or holder 128 of the needle arrangement, cf. FIG. 57) may be advantageous during high-volume manufacture of the catheter or cannula arrangement.

As mentioned before, the aspect concerning the pull-out tab or separating element 35 is not limited to embodiments with caps for closing injection ports of catheter or cannula arrangements. E.g., in an embodiment such as the one of FIGS. 55 to 57 or FIG. 58, it is also possible to provide a separating element 35 (not illustrated), e.g., attached to protruding portion 172 (cf. FIG. 57) which in that case would not need to be provided with a metal coating 22.

Above, we have described various ways of signals which may be emitted by the signalling unit, such as displaying a time numerically, cf. FIG. 58, and signalling a status using one or two light emitters such as LEDs (cf., e.g., FIGS. 3, 4, 18, 19) and transmitting corresponding information via radiofrequency signals to a remote location. However, another useful possibility is to use intensity-modulated light for transmitting information such as the dwell time or a remaining time or a status in encoded form. The information can be encoded in a series of light pulses. A reader with a light detector such as a photodiode can be used for detecting the modulated light and include or be connected to a decoder for decoding the detected light intensity modulation into the desired transmitted (time) information. The reader may have a display for displaying the transmitted information or be operationally connected to another apparatus having such a display.

Of course, numerous variations to the embodiments described in this section are possible and are readily contemplated in view of present disclosure. Various of these have already been described in section “Summary of the Invention” above. For example, instead of providing portions of security units in caps or attachments to caps, it is generally also possible to provide portions of security units elsewhere. And the catheter or cannula arrangement may generally be not provided with an injection port. For example, portions of security units may be provided in a holder or shaft part holding the catheter or cannula and/or be non-removably interconnected to the catheter or cannula. Or, they are provided in or at a valve device, or located in an extension. And, instead of detecting an opening of an electric contact and/or of an electric circuit for detecting that the time of a stay of the catheter or cannula in a body starts, other ways are possible, e.g., by detecting a closing of an electrical contact and/or of an electrical circuit, or a button of the security unit could be pressed instead (further examples have been given above). And, alternatively to or additionally to providing optical signals for indicating a status (e.g., that everything is assumed to be in good order, or that it will soon be or already is time to remove the catheter or cannula from the body), it would also be possible to use, e.g., acoustical signals or it would be possible to indicate the amount of time that has passed since inserting the catheter or cannula or the time remaining until the catheter or cannula should be removed. And, a transmission to an external device (not included to the catheter or cannula arrangement) of data indicative of a sensed physical quantity such as a sensed temperature and/or of data related to the dwell time such as data indicative of the dwell time or data indicative of the time remaining until the end of the preset time span (which usually is the time remaining until the catheter or cannula should be removed from the body) can be provided. Such a transmission may be accomplished in a wirebound fashion, e.g., via a digital interface. But it is also possible to accomplish this in a wireless fashion, typically using a wireless transmitter or transceiver, e.g., in the radiofrequency range. Further examples have been given above.

Some portions of the invention and aspects of the embodiments, respectively, are described in the present patent application in terms of functional units. As is readily understood, these functional units may be realized in virtually any number of hardware and/or software components adapted to performing the specified functions. For example, as explained, integrated circuit 218 (or two or more integrated circuits) may embody timer unit 4 as well as signalling unit 5 as well as a portion of activation detection unit 3.

Providing a catheter or cannula arrangement with a security unit 2 may greatly contribute to avoiding the catheter or cannula 10 staying in a body for too long, in particular by generating warning signals (such as signals S1) indicating that the time for removing the catheter or cannula 10 from the body will soon be reached or has already been reached. And the provision of an electronics unit can facilitate obtaining and/or perceiving sensed data such as a body temperature.

1-88. (canceled)

89. A catheter or cannula arrangement, comprising:
a catheter or cannula;
a security unit for monitoring a length of stay of said catheter or cannula in a body;
wherein said security unit comprises an activation detection unit for detecting a first event and for producing a signal indicative of detection of said first event.

90. The arrangement according to claim 89, wherein said first event is at least one of:
movement of one portion of the arrangement relative to another portion of the arrangement;
removal of one portion of the arrangement from another portion of the arrangement;
application of a force to at least a portion of the arrangement;
deformation of at least a portion of the arrangement.

91. The arrangement according to claim 89, comprising a needle insertable into and extractable from said catheter or cannula, wherein said first event is an at least partial extraction of said needle from said catheter or cannula.

92. The arrangement according to claim 91, wherein said activation detection unit is structured and configured for detecting said at least partial extraction of said needle from said catheter or cannula in at least one of the following ways:
mechanically;
electrically;
via a change of electric resistance;
inductively;
capacitively;
magnetically;
via a change of inductivity;
optically.

93. The arrangement according to claim 92, wherein said activation detection unit comprises a first portion directly or indirectly fixed to said catheter or cannula and a second portion directly or indirectly fixed to said needle.

94. The arrangement according to claim 92, further comprising an injection port and a cap for sealing said injection port, wherein said first portion of said activation detection unit is arranged in said cap or is arranged in an attachment to said cap.

95. The arrangement according to claim 92, comprising a needle arrangement, said needle arrangement comprising said needle, wherein said activation detection unit comprises a first portion directly or indirectly fixed to said catheter or cannula and a second portion directly or indirectly fixed to said needle, and wherein said second portion of said activation detection unit is either:
I) integrated or comprised in said needle arrangement; or
II) integrated in or comprises an attachment to said needle arrangement.

96. The arrangement according to claim 95, wherein said needle arrangement comprises a handle, and, in case I), said second portion of said activation detection unit is integrated or comprised in said handle and, in case II), said second portion of said activation detection unit is integrated or comprised in said attachment to said handle.

97. The arrangement according to claim 95, wherein said second portion of said activation detection unit comprises at least one of:
an electrical contact element; and
a separating element for electrically separating electrical contact elements.

98. The arrangement according to claim 97, wherein said second portion of said activation detection unit comprises a contacting portion in which said electrical contact element is arranged, said electrical contact element being, in case I), integrated in or fixed to said needle arrangement and, in case II), integrated in or fixed to said attachment to said needle arrangement.

99. The arrangement according to claim 98, wherein said second portion of said activation detection unit comprises said electrical contact element.

100. The arrangement according to claim 93, wherein said first and second portions of said activation detection unit each comprise at least one electrical contact element.

101. The arrangement according to claim 100, wherein said electrical contact elements are structured and arranged such that they form a closed electrical contact when said needle is in an inserted position in said catheter or cannula and that they are separated from each other when said needle is extracted from said catheter or cannula.

102. The arrangement according to claim 93, wherein said first portion of said activation detection unit comprises a first and a second electrical contact element, said second portion of said activation detection unit comprising an electrical contact element referred to as third electrical contact element, wherein said first, second and third electrical contact elements are structured and arranged such that:
said third electrical contact element bridges said first and second electrical contact elements when said needle is inserted in said catheter or cannula; and
said third electrical contact element does not bridge said first and second electrical contact elements when said needle is extracted from said catheter or cannula.

103. The arrangement according to claim 101, wherein at least one of:
said first and second contact elements are formed by contact leads or tracks of a printed circuit board; and
said third contact element is formed by a piece of sheet metal.

104. The arrangement according to claim 95, wherein said second portion of said activation detection unit comprises at least one of:
an electrical contact element; and
an element for electrically separating electrical contact elements, referred to as separating element;
and wherein said separating element comprises an electrically insulating portion

105. The arrangement according to claim 104, wherein said electrically insulating portion is made of electrically insulating material.

106. The arrangement according to claim 104, wherein said electrically insulating portion is formed by an electrically insulating foil or by an electrically insulating member having a sheet shape.

107. The arrangement according to claim 104, wherein said security unit comprises a first and a second electrical contact element, and wherein said separating element and said first and second electrical contact elements are structured and arranged such that:
when said needle is inserted in said catheter or cannula, said electrically insulating portion is arranged in a location between said first and second electrical contact elements, electrically separating said first contact element from said second contact element; and
when said needle is extracted from said catheter or cannula, said electrically insulating portion is removed from the location, thereby allowing said first and second contact elements to be in electrical contact with each other.

108. The arrangement according to claim 104, wherein said separating element has a first end and a second end, said electrically insulating portion being located at the first end of said separating element, and said second end of the separating element being fixed, in case I), to said needle arrangement and, in case II), to said attachment to said needle arrangement.

109. The arrangement according to claim 89, wherein said security unit comprises a signalling unit for at least one of:
emitting a first signal indicative of attainment of an end of a preset time span;
indicating a time corresponding to said length of stay or corresponding to a time remaining up to a preset time span.

110. The arrangement according to claim 109, wherein said preset time span is between 2 days and 6 days.

111. The arrangement according to claim 109, wherein said first signal is at least one of
a visual signal;
an optical signal;
an acoustic signal;
an electric signal;
a magnetic signal;
an electromagnetic signal,
an electromagnetic signal in the optical range;
an electromagnetic signal in the radio frequency range;
an electromagnetic signal in the microwave range;
a mechanical signal;
a vibration.

112. A security unit for a catheter or cannula arrangement comprising a catheter or cannula, wherein said security unit is structured and configured for monitoring a length of stay of said catheter or cannula in a body, the security unit comprising:
a signalling unit for emitting a first signal;
an activation detection unit for detecting a first event and for producing a second signal indicative of detection of said first event;
a timer unit for receiving said second signal from said activation detection unit and for starting a time duration measurement in reaction to receiving said second signal.

113. The security unit according to claim 112, wherein said timer unit is provided for producing and transmitting to said signalling unit a third signal as soon as the measured time duration is as large as or exceeds a preset time span, wherein said signalling unit is structured and configured for emitting said first signal in reaction to receiving said third signal.

114. The security unit according to claim 112, comprising a printed circuit board assembly, said printed circuit board assembly comprising at least a portion of said signalling unit, at least a portion of said activation detection unit, at least a portion of said timer unit.

115. The security unit according to claim 112, wherein said activation detection unit comprises electrical contact elements.

116. The security unit according to claim 115, wherein said first event is detected via a change of an electric resistance between two or more of said electrical contact elements.

117. An electronics unit arrangement for a catheter or cannula arrangement comprising a catheter or cannula and a sensing element, the electronics unit arrangement comprising:
an electronics unit for processing signals produced by the sensing element and/or for storing data related to signals produced by the sensing element;
an electrical connector for providing an electrical contact to said sensing element;
a housing containing at least a portion of said electronics unit;
one or more fixing structures for attaching said housing to the catheter or cannula.

118. The electronics unit arrangement according to claim 117, comprising at least one of
a storage unit for storing data related to signals produced by the sensing element;
electronic circuitry for processing signals produced by the sensing element and/or for obtaining data related to signals produced by the sensing element;
a display for displaying said data or other data related to signals produced by the sensing element;
a user control;
an interface, for outputting said data or other data related to signals produced by the sensing element;
a radio frequency transmitter or transceiver for wirelessly transmitting said data or other data related to signals produced by the sensing element.

119. The electronics unit arrangement according to claim 117, further comprising an opening and a contacting element or a separating element extending through said opening operationally connected to at least one electric contact element of the electronics unit.
